(19) United States
(12) Patent Application Publication
(10) Pub. No.: US 2026/0115299 A1
(43) Pub. Date: Apr. 30, 2026

(54) CALSEQUESTRIN-BASED METAL ION REACTIVE PARTICLE AND USES THEREOF

(71) Applicant: TOOLBIO. CO.,LTD., Daejeon (KR)

(72) Inventors: Se Ho PARK, Sejong (KR); Sung Hyun KIM, Sejong (KR)

(21) Appl. No.: 18/839,807

(22) PCT Filed: Feb. 21, 2023
(86) PCT No.: PCT/KR2023/002458

§ 371 Date: Aug. 20, 2024

(30) Foreign Application Priority Data
Feb. 21, 2022 (KR) 10-2022-0022228
Feb. 21, 2023 (KR) 10-2023-0022644

(51) Int. Cl. A61K 47/64; A61K 35/14; A61K 38/17; A61K 38/18; A61K 38/19; A61K 38/22; A61K 38/43; A61K 39/00; A61K 47/02; A61K 47/69; C07K 16/00

(52) U.S. Cl. CPC A61K 47/64; A61K 35/14; A61K 38/1741; A61K 38/18; A61K 38/19; A61K 38/22; A61K 38/43; A61K 39/00; A61K 47/02; A61K 47/6929; C07K 16/00

(57) ABSTRACT

The present invention relates to calsequestrin-based metal ion reactive particles and their applications. More specifically, the invention concerns calsequestrin-based metal ion reactive particles, which are prepared by combining bioactive substances frequently used in pharmaceuticals and cosmetics with calsequestrin (CSQ) and then reacting them with metal ions. This invention also relates to the use of these particles as drug delivery carriers, pharmaceutical compositions, or vaccines. The metal ion reactive particles according to the present invention can enhance the in vivo and in vitro stability of bioactive substances, prolong their active duration, increase their half-life in the body, and improve antigen delivery efficiency.

### TECHNICAL FIELD

The present invention relates to calsequestrin-based metal ion-responsive particles and their uses. More specifically, the present invention relates to calsequestrin (CSQ)-based metal ion-responsive particles prepared by conjugating bioactive substances, which are widely used in pharmaceuticals and cosmetics, with calsequestrin and subsequently reacting them with metal ions, and their uses in drug delivery vehicles, pharmaceutical compositions, or vaccines.

The instant application contains a Sequence Listing which has been filed electronically in ASCII format and is hereby incorporated by reference in its entirety. Said ASCII copy, created on Apr. 9, 2025, is named 77265-00004-PCT.xml and is 32,768 bytes in size.

### BACKGROUND ART

Protein and peptide drugs, when administered orally, generally lose their active structure in the acidic environment of the stomach or are enzymatically degraded, resulting in low absorption rates in the stomach or intestines. Even when administered parenterally, these drugs have a short half-life in vivo or low bioavailability, leading to ongoing research on sustained release of protein and peptide drugs to achieve prolonged therapeutic effects. Furthermore, even for synthetic drugs, research on drug delivery systems that enable slow and sustained release in vivo is ongoing. In this context. Korean Patent Publication No. 10-2020-0044977 discloses microparticles containing a biodegradable polymer and a drug. Meanwhile, calsequestrin is a calcium-binding protein in the sarcoplasmic reticulum that functions to store calcium ions in the cisternae of the sarcoplasmic reticulum by binding to calcium after muscle contraction, even though the concentration of calcium in the sarcoplasmic reticulum is higher than in the cytoplasm. In this case, calsequestrin exists as a monomer when calcium is low but changes to a larger multimeric form as the amount of calcium increases.

### PRIOR ART DOCUMENT

Korean Patent Publication No. 10-2020-0044977

### DETAILED DESCRIPTION OF THE INVENTION

### Technical Problem

The inventors of the present invention have made extensive efforts to develop a drug delivery system using novel materials. As a result, they found that when bioactive substances (proteins, peptides, or low molecular weight drugs) are conjugated with calsequestrin and formulated with metal ions, the stability of these substances in vitro and in vivo is enhanced, their in vivo half-life is increased, antigen delivery efficiency is improved, and the in vivo effects are sustained. Thus, the present invention has been completed.

The present invention aims to provide particles comprising calsequestrin conjugated with bioactive substances, their uses, and methods for their preparation.

The present invention aims to provide particles comprising calsequestrin conjugated with bioactive substances to increase the stability of bioactive substances administered in vivo and methods for their preparation.

The present invention aims to provide particles comprising calsequestrin conjugated with bioactive substances to increase the half-life of bioactive substances administered in vivo and methods for their preparation.

The present invention aims to provide particles comprising calsequestrin conjugated with bioactive substances to increase the sustainability of the in vivo effects of bioactive substances administered in vivo and methods for their preparation.

The present invention aims to provide particles comprising calsequestrin conjugated with antigen substances to increase the efficiency of antigen delivery administered in vivo and methods for their preparation.

### Technical Solution

According to the first embodiment,

Particles comprising:
calsequestrin,
a bioactive substance conjugated to the calsequestrin, and
a metal ionically bound to the calsequestrin are disclosed.

In the present invention, the metal can be selected from the group consisting of Cu, Ra, Ba, Sr, Ca, Cd, Co, Cr, Cu, Be, Fe(II), Zn, Mg, Mn, Co, Ni, Al, Fe(III), Au, He, Cr, Si, V, Ga, In, La, and Ce.

In the present invention, the metal may exclude calcium.

In the present invention, the metal may include calcium.

In the present invention, the metal may include zinc.

In the present invention, the metal may include magnesium.

In the present invention, the particles are calsequestrin-based metal ion-responsive particles comprising bioactive substances fused with calsequestrin, and the particles can self-assemble by calcium.

In the present invention, the bioactive substance may be covalently bonded to calsequestrin.

In the present invention, the bioactive substance may not be covalently bonded to calsequestrin.

In the present invention, the bioactive substance linked to calsequestrin may include a cleavage site between the bioactive substance and calsequestrin.

In the present invention, the cleavage site between the bioactive substance and calsequestrin may be cleaved by protease, nuclease, lipase, glycosidase, pH, 5hypoxia, light, heat, enzyme, ultrasound, x-ray, or hydrolysis.

In the present invention, the bioactive substance conjugated to calsequestrin may not be cleaved.

In the present invention, multiple calsequestrin units can bind to a single metal.

In the present invention, the calsequestrin may include one or more amino acid sequences selected from the group consisting of SEQ ID NO: 1 to 11, having at least 85% sequence homology.

In the present invention, the calsequestrin may have a partially modified C-terminal amino acid sequence of wild-type calsequestrin.

In the present invention, the calsequestrin may have a partial deletion of the C-terminal amino acid sequence of wild-type calsequestrin.

In the present invention, the calsequestrin may include a sequence selected from the group consisting of SEQ ID NO: 1 to 11.

In the present invention, the calsequestrin may be composed of a sequence selected from the group consisting of SEQ ID NO: 1 to 11.

In the present invention, the particles may exclude elastin-like polypeptides or elastomer.

In the present invention, the particles may exclude sequences with repeating units of VPGXG (where X is an amino acid).

In the present invention, the bioactive substance may be selected from the group consisting of polymeric proteins, peptides, glycoproteins, cytokines, growth factors, blood preparations, vaccines, hormones, enzymes, antibodies, and synthetic agents.

In the present invention, the bioactive substance may be selected from the group consisting of interleukin-2, blood factor VII, blood factor VIII, blood factor IX, immunoglobulin, horseradish peroxidase (HRP), cytokines, α-interferon, β-interferon, γ-interferon, colony-stimulating factor (GM-CSF), human fibronectin extra domain B (EBD), fibroblast growth factor (FGF), nerve growth factor (NGF), insulin-like growth factor (IGF), transforming growth factor-α and -β (TGF-α, -β), brain-derived neurotrophic factor (BDNF), platelet-derived growth factor (PDGF), placental growth factor (PlGF), hepatocyte growth factor (HGF), exendin, somatostatin, luteinizing hormone-releasing hormone (LHRH), adrenocorticotropic hormone (ACTH), growth hormone-releasing hormone (GHRH), oxytocin, thymosin alpha-1, corticotropin-releasing factor (CRF), calcitonin, bivalirudin, vasopressin, phospholipase-activating protein (PLAP), insulin, tumor necrosis factor (TNF), follicle-stimulating hormone (FSH), thyroid-stimulating hormone (TSH), antidiuretic hormone (ADH), melanocyte-stimulating hormone (MSH), parathyroid hormone (PTH), luteinizing hormone (LH), calcitonin gene-related peptide (CGRP), enkephalin, somatomedin, erythropoietin, hypothalamic-releasing factor, prolactin, chronic gonadotropin, tissue plasminogen activator, growth hormone-releasing peptide (GHRP), thymic humoral factor (THF), asparaginase, arginase, arginine deaminase, adenosine deaminase, superoxide dismutase, endotoxinase, catalase, chymotrypsin, lipase, uricase, adenosine diphosphatase, tyrosinase, bilirubin oxidase, glucose oxidase, glucosidase, galactosidase, glucocerebrosidase, glucuronidase, keratinocyte growth factor (KGF), exenatide, doxorubicin, anti-VEGF scFv, SARS Cov2 RBD (receptor binding domain), epidermal growth factor (EGF), fibroblast growth factor-2 (FGF-2), bone morphogenetic protein (BMP2), transforming growth factor (TGF), vascular endothelial growth factor (VEGF), glucagon-like peptide-1 (GLP-1). TEV protease, HRP enzyme, human growth hormone (hGH), and hematopoietic growth factor (G-CSF).

In the present invention, the bioactive substance may be an interest polypeptide.

In the present invention, the bioactive substance may have a length of 2 to 1000 amino acids.

In the present invention, the bioactive substance may be a chemical substance of interest.

In the present invention, the bioactive substance may be selected from the group consisting of immunomodulatory compounds, anticancer agents, antiviral agents, antibacterial agents, antifungal agents, and antiparasitic agents.

In the present invention, the average diameter of the particles may range from 10 nm to 1 cm.

In the present invention, the particles may not be assembled by temperature changes.

In the present invention, the particles may exclude polymers.

In the present invention, the particles may be used to deliver bioactive substances to a target requiring them.

In the present invention, the particles may be used for tissue regeneration.

In the present invention, the half-life of the bioactive substances may be increased compared to before the bioactive substances are formulated into particles.

In the present invention, the half-life of the bioactive substances may be increased by at least 10% compared to before the bioactive substances are formulated into particles.

In the present invention, the in vivo stability of the bioactive substances may be increased compared to before the bioactive substances are formulated into particles.

In the present invention, the in vivo stability of the bioactive substances may be increased by at least 10% compared to before the bioactive substances are formulated into particles.

In the present invention, the sustained effect of the bioactive substances may be increased compared to before the bioactive substances are formulated into particles, as the in vivo stability of the bioactive substances increases.

### Second Embodiment

According to the second embodiment, a pharmaceutical composition for the prevention or treatment of one or more diseases selected from the group consisting of skin wounds, diabetes, inflammatory diseases, ophthalmic diseases, tissue regeneration, infectious diseases, and cancer, comprising the particles according to the first embodiment, is disclosed.

In the present invention, the composition may further comprise a pharmaceutically acceptable carrier or excipient.

### Third Embodiment

According to the third embodiment, a vaccine for the prevention or treatment of one or more diseases selected from the group consisting of inflammatory diseases, infectious diseases, and cancer, comprising the particles according to the first embodiment or the pharmaceutical composition according to the second embodiment, is disclosed.

In the present invention, the vaccine may include an adjuvant.

In the present invention, the vaccine may be lyophilized

### Fourth Embodiment

According to the fourth embodiment, a kit for manufacturing particles comprising calsequestrin and a bioactive substance configured to be linked to the calsequestrin is disclosed.

In the present invention, the kit may further comprise a metal configured to ionically bind with the calsequestrin.

In the present invention, the kit may be a calsequestrin-based metal ion-reactive particle manufacturing kit comprising calsequestrin and a bioactive substance configured to be linked to the calsequestrin.

In the present invention, the kit may include one or more selected from the group consisting of Cu, Ra, Ba, Sr, Ca, Cd, Co, Cr, Cu, Be, Fe(II), Zn, Mg, Mn, Co, Ni, Al, Fe(III), Au, He, Cr, Si, V, Ga, In, La, and Ce.

In the present invention, the bioactive substance fused with the calsequestrin may be included in the form of nucleotide molecules coating it.

In the present invention, the bioactive substance fused with the calsequestrin may be included in the form of an expression vector containing nucleic acid coating it.

In the present invention, the bioactive substance fused with calsequestrin may be included in the form of cells transformed with an expression vector containing nucleic acid coding for it.

### Fifth Embodiment

According to the fifth embodiment, a method for manufacturing the particles according to the first embodiment is disclosed.

In the present invention, the method may comprise a step of mixing the bioactive substance linked to calsequestrin with a metal.

In the present invention, the method may further comprise a step of linking calsequestrin to the bioactive substance.

In the present invention, the method may further comprise a step of recombinantly expressing a sequence comprising calsequestrin and the bioactive substance.

In the present invention, the method may further comprise a step of recombinantly expressing a sequence comprising calsequestrin and the bioactive substance.

In the present invention, the method may further comprise a step of purifying the bioactive substance linked to calsequestrin.

In the present invention, the method may include:
A) preparing an expression vector comprising nucleic acid coding for a bioactive substance linked to calsequestrin;
B) transforming a host cell with the expression vector to obtain a transformant;
C) expressing a protein comprising the bioactive substance linked to calsequestrin from the transformant; and
D) formulating the expressed protein comprising the bioactive substance linked to calsequestrin into particles using metal ions.

In the present invention, the method may include the host cell being selected from the group consisting of Escherichia coli, Bacillus subtilis, Bacillus thuringiensis, Salmonella typhimurium, Serratia marcescens. Pseudomonas species, yeast, insect cells, Chinese hamster ovary (CHO) cell line, W138, BHK, COS-7, 293, HepG2, 3T3, RIN, MDCK cell line, and plant cells.

In the present invention, the method may include:
A) reacting calsequestrin with a bioactive substance to form a chemical conjugation; and
B) particleizing the bioactive substance conjugated with calsequestrin using metal ions.

In the present invention, the method may further include a step of preparing the bioactive substance to form a chemical conjugation with calsequestrin.

### Sixth Embodiment

According to the sixth embodiment, the present invention discloses a method for delivering a bioactive substance to a subject in need thereof.

In the present invention, the method may include:
manufacturing particles according to the method of the fifth embodiment; and
administering the particles to the subject.

In the present invention, the method may include administering calsequestrin, a bioactive substance, and a metal to the subject to form particles according to the first embodiment.

In the present invention, the calsequestrin may be linked to the bioactive substance.

In the present invention, the administration may include expressing nucleotide molecules encoding calsequestrin and the bioactive substance in the subject.

In the present invention, the administration may include expressing a first nucleotide molecule encoding calsequestrin and a second nucleotide molecule encoding the bioactive substance in the subject.

In the present invention, the administration may include parenteral, oral, subcutaneous, topical, intramuscular, transdermal, buccal, sublingual, intranasal, intravascular, subcutaneous, ocular, or respiratory administration.

In the present invention, the bioactive substance may be administered directly to the target site of the disease in the subject.

In the present invention, the bioactive substance may not be administered directly to the target site of the disease in the subject.

In the present invention, the method may further include the step of releasing the bioactive substance from the particles.

In the present invention, the method may further include the step of releasing the bioactive substance in a free form from the particles.

In the present invention, the method may further include the step of releasing the bioactive substance from the particles while linked to calsequestrin.

According to the seventh embodiment, the use of a product or method characterized by one or more elements disclosed in this application is disclosed.

### Effects of the Invention

The calsequestrin-based metal ion-reactive particles of the present invention can improve stability by inhibiting degradation by hydrolytic enzymes in the body compared to before being particleized by metal ions.

Additionally, the calsequestrin-based metal ion-reactive particles of the present invention can enhance the duration of the bioactive substance's effect by increasing its half-life in the body compared to before being particleized by metal ions.

Moreover, the calsequestrin-based metal ion-reactive particles of the present invention can maintain the activity of the bioactive substance by improving its stability outside the body compared to before being particleized by metal ions.

Furthermore, the calsequestrin-based metal ion-reactive particles of the present invention can increase the delivery efficiency of antigens compared to before being particleized by metal ions.

### BRIEF DESCRIPTION OF DRAWINGS

FIG. 1 is a schematic diagram showing that, in an exemplary embodiment, the stability, prolonged activity duration, and increased half-life in the body of bioactive substances can be enhanced when CSQ-KGF is particleized by metal ions.

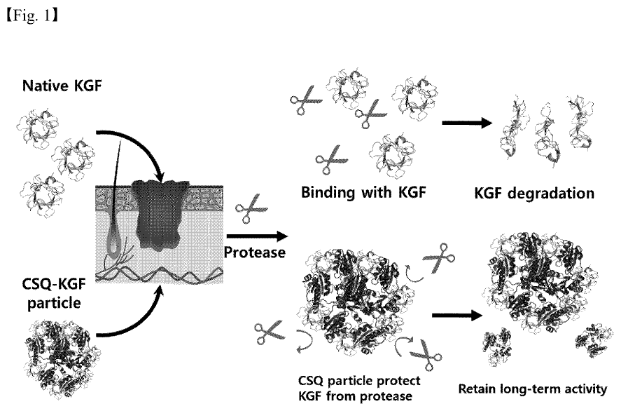

FIG. 2 is a schematic diagram of the CSQ-KGF expression vector.

FIG. 3 shows the SDS-PAGE results of purified CSQ-KGF.

FIG. 4 presents the fluorescence microscopy observation results and the particle distribution (DLS) results of CSQ-KGF particleized by metal ions (Ca²⁺).

FIG. 5 shows the stability evaluation data, as indicated by SDS-PAGE results, of CSQ-KGF particleized by various metal ions (Ca²⁺, Mg²⁺, Fe²⁺, Zn²⁺, Cu²⁺, and Mn²⁺).

FIG. 6 illustrates the release rate evaluation results of CSQ-KGF particleized by various metal ions (Ca²⁺, Mg²⁺, Fe²⁺, Zn²⁺, Cu²⁺, and Mn²⁺).

FIG. 7 presents the in vitro proliferation test results of CSQ-KGF, CSQ, and native KGF.

FIG. 8 shows the in vitro migration test results of CSQ-KGF. CSQ, and native KGF.

FIG. 9 displays the in vivo wound healing rate evaluation results of the control group (Saline), native KGF, and CSQ-KGF particleized by metal ions (Ca²⁺).

FIG. 10 presents the wound suture analysis results of CSQ-KGF particleized by metal ions (Ca²⁺) and non-particleized CSQ-KGF (No particle).

FIG. 11 shows a schematic diagram of the Exenatide-CSQ expression vector (A) and the SDS-PAGE results of purified CSQ-KGF (B).

FIG. 12 shows the turbidity and particle distribution (DLS) results of Exenatide-CSQ particleized by metal ions (Ca²⁺).

FIG. 13 presents the stability evaluation data of Exenatide-CSQ particleized by various metal ions (Ca²⁺, Mg²⁺, Fe²⁺, Zn²⁺, Cu²⁺, and Mn²⁺), including the SDS-PAGE results (A), stability evaluation based on metal ion concentration (B), and stability evaluation based on the type of metal ion (C).

FIG. 14 illustrates the in vivo blood glucose reduction rate evaluation results of native Exenatide, Exenatide-CSQ particleized by metal ions (Ca²⁺), and non-particleized Exenatide-CSQ (No particle).

FIG. 15 shows the in vivo half-life evaluation results of native Exenatide. Exenatide-CSQ particleized by metal ions (Ca²⁺), and non-particleized Exenatide-CSQ (No particle).

FIG. 16 presents the particleization results of CSQ-SARS Cov2 RBD based on metal ion (Ca²⁺) concentration (A) and the evaluation results of immunogenic vaccine efficacy (B).

FIG. 17 is a schematic diagram showing the method of preparing CSQ-Doxorubicin particleized by metal ions.

FIG. 18 displays the particle distribution (DLS) results of CSQ-Doxorubicin particleized by metal ions (Ca²⁺).

FIG. 19 presents the in vivo half-life evaluation results of native Doxorubicin, CSQ-Doxorubicin particleized by metal ions (Ca²⁺), and non-particleized CSQ-Doxorubicin (No particle).

FIG. 20 shows the particle distribution (DLS) results of CSQ-EGF particleized by metal ions (Ca²⁺).

FIG. 21 shows the particle distribution (DLS) results of CSQ-Anti-VEGF scFv particleized by metal ions (Ca²⁺).

FIG. 22 shows the particle distribution (DLS) results of CSQ-FGF2 particleized by metal ions (Ca²⁺).

FIG. 23 shows the particle distribution (DLS) results of CSQ-BMP2 particleized by metal ions (Ca²⁺).

FIG. 24 shows the particle distribution (DLS) results of CSQ-TGF particleized by metal ions (Ca²⁺).

FIG. 25 shows the particle distribution (DLS) results of CSQ-VEGF particleized by metal ions (Ca²⁺).

FIG. 26 shows the particle distribution (DLS) results of CSQ-GLP1 particleized by metal ions (Ca²⁺).

FIG. 27 shows the particle distribution (DLS) results of CSQ-TEV protease particleized by metal ions (Ca²⁺).

FIG. 28 shows the particle distribution (DLS) results of CSQ-HRP enzyme particleized by metal ions (Ca²⁺).

FIG. 29 presents the stability evaluation results of CSQ-HRP enzyme particleized by metal ions (Ca²⁺) and non-particleized CSQ-HRP enzyme (No particle).

FIG. 30 shows the particle distribution (DLS) results of CSQ-hGH particleized by metal ions (Ca²⁺).

FIG. 31 shows the particle distribution (DLS) results of CSQ-hGH particleized by various metal ions (Ca²⁺, Mg²⁺, Fe²⁺, Zn²⁺, Cu²⁺, Mn²⁺).

FIG. 32 shows the particle distribution (DLS) results of CSQ-GCSF particleized by metal ions (Ca²⁺).

FIG. 33 shows the amino acid sequences of CSQ2 WT, CSQ2 Δ8, and CSQ2 Δ16.

FIG. 34 presents the absorbance results (A) and particle distribution (DLS) results (B) of CSQ2 (WT), CSQ2 (Δ8), and CSQ2 (Δ16) particleized by metal ions (Ca²⁺).

FIG. 35 shows the particle distribution (DLS) results of KGF-CSQ2 (WT), KGF-CSQ2 (Δ8), and KGF-CSQ2 (Δ16) particleized by metal ions (Ca²⁺).

FIG. 36 presents the SDS-PAGE results of KGF separated from CSQ-cleavable linker-KGF particleized by metal ions (Ca²⁺).

FIG. 37 presents the SDS-PAGE results of hGH separated from CSQ-cleavable linker-hGH particleized by metal ions (Ca²⁺).

FIG. 38 shows the particle distribution (DLS) results of Exenatide-CSQ conjugation particleized by metal ions (Ca²⁺).

FIG. 39 shows the particle distribution (DLS) results of hGH-CSQ conjugation particleized by metal ions (Ca²⁺).

### BEST MODE FOR CARRYING OUT THE INVENTION

Unless otherwise defined, all technical and scientific terms used herein have the same meaning as commonly understood by one of ordinary skill in the art to which this disclosure belongs. Any methods, materials, compositions, reagents, and cells similar or equivalent to those described herein can be used in the practice or testing of the subject matter of the present disclosure, although preferred methods and materials are described. All publications and references cited herein, including patents and patent applications, are incorporated herein by reference in their entirety as if each individual publication or reference were specifically and individually indicated to be incorporated by reference. Any patent application to which this application claims priority is also incorporated by reference in its entirety in the manner described for publications and references.

As used herein, the term “amino acid” refers to both natural and non-natural amino acids, as well as amino acid analogs and mimetics. Natural amino acids include the 20 (L)-amino acids used in protein biosynthesis, as well as others such as 4-hydroxyproline, hydroxylysine, desmosine, isodesmosine, homocysteine, citrulline, and ornithine. Non-natural amino acids include, for example, (D)-amino acids, norleucine, norvaline, p-fluorophenylalanine, and ethionine, which are known to those skilled in the art. Amino acid analogs include modified forms of natural and non-natural amino acids. Such modifications may include, for example, substitution or replacement of chemical groups or moieties on the amino acids, or derivatization of the amino acids. Amino acid mimetics include organic structures that exhibit functionally similar properties, such as charge and charge spacing, to those of standard amino acids. For example, an organic structure that mimics arginine (Arg or R) would have a positively charged moiety in a similar molecular space and with similar mobility as the ε-amino group of the side chain of natural Arg. Mimetics may also include constrained structures that maintain the optimal spacing and charge interactions of amino acids or amino acid functional groups.

A person skilled in the art will know or be able to determine the structures that constitute functionally equivalent amino acid analogs and mimetics.

The term “elastin-like polypeptide,” as used herein, refers to a type of amino acid polymer that undergoes conformational changes in response to temperature. These elastin-like polypeptides may be polymers exhibiting “inverse phase transitioning behavior.” Inverse phase transitioning behavior refers to the property where the polymer is soluble in aqueous solution below its “inverse phase transition temperature (Tt).” and becomes insoluble when the temperature rises above Tt. The elastin-like polypeptide can transition from a highly soluble elongated chain at lower temperatures to a tightly folded aggregate with significantly reduced solubility as the temperature increases. This inverse phase transition can be induced by the elastin-like polypeptide adopting more β-turn and distorted β-structure configurations as the temperature rises. These elastin-like polypeptides may have phase transition temperatures ranging, for example, from about 10° C. to about 70° C. or from about 39° C. to about 70° C.

The term “half-life,” as used herein, may refer to the time it takes for a bioactive substance according to the present invention to lose half of its pharmacological, physiological, or other activity when administered to an organism's serum or tissue compared to the initial activity or any other defined point in time. The term “half-life” may also refer to the time it takes for the amount or concentration of the bioactive substance according to the present invention to decrease to half of the initial administered amount or concentration in the organism's serum or tissue compared to the starting amount or any other defined point in time. The half-life can be measured in serum and/or any one or more selected tissues.

The terms “modulating” and “changing” as used herein include both “increasing,” “enhancing.” or “stimulating” as well as “decreasing” or “diminishing.” typically in a statistically significant or physiologically meaningful amount or degree compared to a control. An “increased.” “stimulated.” or “enhanced” amount is typically a “statistically significant” amount, which may include an increase of 1.1, 1.2, 1.5, 2, 3, 4, 5, 6, 7, 8, 9, 10, 15, 20, 30, 40, 50, 60, 70, 80, 90, 100-fold or more (e.g., 500, 1000-fold) over the amount produced by a control composition or the absence of calreticulin and its ionically bound metal.

The terms “polypeptide,” “protein,” and “peptide” are used interchangeably and refer to a polymer of amino acids that is not limited by any specific length. The term “polypeptide” or “protein” refers to a chain of one or more amino acids, where each chain is covalently linked by peptide bonds. This polypeptide or protein may include a sequence derived from natural proteins, i.e., peptides linked non-covalently and/or covalently by peptide bonds, which may be produced by natural, non-recombinant cells, or genetically modified or recombinant cells, and may also include molecules with sequences of natural proteins, or molecules with deletions, additions, and/or substitutions from one or more amino acids of a natural sequence. In certain embodiments, the polypeptide may be a “recombinant” polypeptide produced by recombinant cells containing one or more recombinant DNA molecules, which typically includes heterologous polynucleotide sequences or combinations of polynucleotide sequences not otherwise found in the cell.

The terms used in this specification have the following meanings:

“Polynucleotide” and “nucleic acid” refer to mRNA, RNA, CRNA, cDNA, and DNA. These terms typically denote polymers of nucleotides with a length of at least 10 nucleotides, which may be modified forms of ribonucleotides or deoxynucleotides. The terms encompass both single-stranded and double-stranded DNA.

“Isolated DNA,” “isolated polynucleotide,” and “isolated nucleic acid” refer to molecules that are isolated from the genomic DNA of a particular species. For instance, an isolated DNA segment encoding a polypeptide contains one or more coding sequences but is substantially isolated or purified from the genomic DNA of the species from which it is obtained. This also includes non-coding polynucleotides (e.g., primers, probes, oligonucleotides). Recombinant vectors, such as expression vectors, viral vectors, plasmids, cosmids, phagemids, phages, and viruses, are also included.

“Drug delivery system” refers to systems for delivering a therapeutically effective drug to the desired location in a living organism. This includes drug compositions, drug formulations, formulation methods, or drug products that ensure efficient delivery of the drug to the required tissues and in the necessary amounts.

“Effective ingredient” refers to a component that, either alone or in combination with a carrier, exhibits the desired activity. It may refer to an ingredient that shows activity on its own or one that, when combined with a carrier, demonstrates the intended effect.

“Prevention” refers to any action that suppresses or delays the onset of a disease through the administration of a composition as described in the invention.

“Treatment” refers to any action that improves or beneficially modifies the symptoms caused by a disease through the administration of the composition described in the invention.

The term “fusion” as used in this specification refers to a form where two or more proteins are artificially linked together. In the context of the present invention, it denotes a protein in which calsequestrin is linked with a bioactive substance. Such fusion proteins can be obtained through chemical conjugation or by expression and purification using recombinant DNA methods. There are no specific limitations on the sequence in which calsequestrin and the bioactive substance are linked in the fusion protein: thus, both “N-terminal-calsequestrin-bioactive substance-C-terminal” and “N-terminal-bioactive substance-calsequestrin-C-terminal” forms are included.

### I. Particles

The present invention provides particles comprising:
calsequestrin,
A bioactive substance conjugated with the calsequestrin, and
A metal ionically bonded to the calsequestrin.

In the particles according to the present invention, the metal may be one or more selected from the group consisting of Cu, Ra, Ba, Sr, Ca, Cd, Co, Cr, Be, Fe(II), Zn, Mg, Mn, Ni, Al, Fe(III), Au, He, Si, V, Ga, In, La, and Ce, but is not limited to these. As an illustrative example, metals such as Ca, Mg, Fe, Zn, Cu, and Mn have been used.

In the particles according to the present invention, the metal may exclude calcium.

In the particles according to the present invention, the metal may include calcium.

In the particles according to the present invention, the metal may include zinc.

In the particles according to the present invention, the metal may include magnesium.

In the particles according to the present invention, the particles are calsequestrin-based metal ion-responsive particles that include a bioactive substance fused with calsequestrin, and may self-assemble due to calcium.

In the particles according to the present invention, the bioactive substance may be covalently bonded to the calsequestrin.

In the particles according to the present invention, the bioactive substance may not be covalently bonded to the calsequestrin.

In the particles according to the present invention, the bioactive substance linked to the calsequestrin may include a cleavage site between the bioactive substance and the calsequestrin. In the particles according to the present invention, the bioactive substance linked to the calsequestrin may include a cleavage site between the bioactive substance and the calsequestrin, where the cleavage site can be cleaved by an enzyme, pH, or hydrolysis.

In the particles according to the present invention, the bioactive substance linked to the calsequestrin may include a cleavable linker between the bioactive substance and the calsequestrin, where the cleavable linker may be selected from the group consisting of protease-cleavable peptide linkers, nuclease-sensitive nucleic acid linkers, lipase-sensitive lipid linkers, glycosidase-sensitive carbohydrate linkers, pH-sensitive linkers, hypoxia-sensitive linkers, photo-cleavable linkers, heat-unstable linkers, enzyme-cleavable linkers, ultrasound-sensitive linkers, and X-ray cleavable linkers, but is not limited to these.

For example, in the case of the protease-cleavable peptide linker, the protease may include metalloproteases, serine proteases, cysteine proteases, aspartic proteases, and the like, but is not limited to these. In this case, the protease-cleavable peptide linker may be cleaved by one or more proteases selected from the group consisting of MMP1, MMP2, MMP3. MMP4. MMP5, MMP6, MMP7, MMP8, MMP9, MMP10, MMP11, MMP12, MMP13, MMP14, TEV protease, matriptase, uPA, FAP, legumain, PSA, kallikrein, cathepsin A, and cathepsin B. In an exemplary embodiment, the protease-cleavable peptide linker may be a PLGVRG linker cleavable by MMP2.

In the particles according to the present invention, the bioactive substance conjugated to the calsequestrin may not be cleaved between the bioactive substance and the calsequestrin.

In the particles according to the present invention, the bioactive substance linked to the calsequestrin may include a non-cleavable linker between the bioactive substance and the calsequestrin.

In the particles according to the present invention, multiple calsequestrins may be bound to a single metal.

In the particles according to the present invention, the amino acid sequence of the calsequestrin may include one or more sequences selected from the group consisting of amino acid sequences numbered 1 to 11, with at least 85%, 86%, 87%, 88%, 89%, 90%, 91%, 92%, 93%, 94%, 95%, 96%, 97%, 98%, or 99% sequence identity.

In the particles according to the present invention, the amino acid sequence of the calsequestrin may include one or more sequences selected from the group consisting of amino acid sequences numbered 1 to 11.

In the particles according to the present invention, the amino acid sequence of the calsequestrin may be composed of one or more sequences selected from the group consisting of amino acid sequences numbered 1 to 11.

In the particles according to the present invention, the calsequestrin may be a wild-type calsequestrin or a variant form in which part of the C-terminal sequence of the wild-type calsequestrin has been modified. The modified form may be a variant where part of the C-terminal amino acid sequence of the wild-type calsequestrin is deleted. The modified form may be a variant where the C-terminal acidic tail of the amino acid sequence is deleted. For example, the modified form may be a variant in which 6 to 20 amino acids at the C-terminal of the wild-type calsequestrin amino acid sequence are deleted. In an exemplary embodiment, it has been confirmed that when the acidic tail at the C-terminal of the calsequestrin amino acid sequence is deleted, the particles can be formed even with low concentrations of metal ions.

In the particles according to the present invention, the particles may exclude elastin-like polypeptides or elastomeric polymers. The elastin-like polypeptides may include one or more repeating units selected from the group consisting of VPGXG, PGXGV, GXGVP, XGVPG, GVPGX, and combinations thereof, where V is valine. P is proline, G is glycine, and X is a natural or non-natural amino acid. Each X in the repeating unit may be the same or a different amino acid. The selected repeating units may be repeated two or more times, for example, 2 to 200 times.

In the particles according to the present invention, the particles may exclude sequences with repeating units of VPGXG (where X is an amino acid).

In the particles according to the present invention, the bioactive substance may be one or more selected from the group consisting of polymeric proteins, peptides, glycoproteins, cytokines, growth factors, blood products, vaccines, hormones, enzymes, antibodies, and synthetic agents. For example, the bioactive substance may be one or more selected from the group consisting of interleukin-2, blood factors VII, VIII, and IX, immunoglobulins, horseradish peroxidase (HRP), cytokines, α-interferon, β-interferon, γ-interferon, granulocyte-macrophage colony-stimulating factor (GM-CSF), human fibronectin extra domain B (EBD), fibroblast growth factor (FGF), nerve growth factor (NGF), insulin-like growth factor (IGF), transforming growth factor-α, and -β (TGF-α, TGF-β), brain-derived neurotrophic factor (BDNF), platelet-derived growth factor (PDGF), placental growth factor (PlGF), hepatocyte growth factor (HGF), exenatide, somatostatin, luteinizing hormone-releasing hormone (LHRH), adrenocorticotropic hormone, growth hormone-releasing hormone, oxytocin, thymosin alpha-1, corticotropin-releasing factor, calcitonin, bivalirudin, vasopressin, phospholipase-activated protein (PLAP), insulin, tumor necrosis factor (TNF), follicle-stimulating hormone, thyroid-stimulating hormone, antidiuretic hormone, melanocyte-stimulating hormone, parathyroid hormone, luteinizing hormone, calcitonin gene-related peptide (CGRP), enkephalin, somatomedin, erythropoietin, hypothalamic releasing factors, prolactin, chronic gonadotropin, tissue plasminogen activator, growth hormone-releasing peptide (GHPR), thymic humoral factor (THF), asparaginase, arginase, arginine deaminase, adenosine deaminase, superoxide dismutase, endotoxinase, catalase, chymotrypsin, lipase, uricase, adenosine diphosphatase, tyrosinase, bilirubin oxidase, glucose oxidase, glucodase, galactosidase, glucocerebrosidase, glucuronidase, keratinocyte growth factor (KGF), exenatide, doxorubicin, anti-vascular endothelial growth factor (Anti-VEGF scFv), SARS-COV-2 receptor-binding domain (RBD), epidermal growth factor (EGF), fibroblast growth factor-2 (FGF-2), bone morphogenetic protein-2 (BMP2), transforming growth factor (TGF), vascular endothelial growth factor (VEGF), glucagon-like peptide-1 (GLP-1), TEV protease, HRP enzyme, human growth hormone (hGH), and granulocyte-colony stimulating factor (G-CSF), but is not limited to these. In the particles according to the present invention, the bioactive substance may be a polypeptide of interest.

In the particles according to the present invention, the bioactive substance may have a length of 2 to 1000 amino acids.

In the particles according to the present invention, the bioactive substance may be a chemical of interest.

In the particles according to the present invention, the bioactive substance may be one or more selected from the group consisting of reverse modulators, anticancer agents, antiviral agents, antibacterial agents, antifungal agents, and anthelmintics. The anticancer agents may include, but are not limited to, maytansinoids, auristatins (including MMAE, MMAF), aminopterin, actinomycin, bleomycin, talisomycin, camptothecin, N8-acetylspermidine, 1-(2-chloroethyl)-1,2-dimethylsulfonylhydrazide, esperamicin, etoposide, 6-mercaptopurine, dolastatin, trichothecenes, calicheamicin, taxol, taxane, paclitaxel, docetaxel, methotrexate, vincristine, vinblastine, doxorubicin, melphalan, mitomycin A, mitomycin C, chlorambucil, duocarmycin, L-asparaginase, mercaptopurine, thioguanine, hydroxyurea, cytarabine, cyclophosphamide, ifosfamide, nitrosourea, cisplatin, carboplatin, mitomycin, dacarbazine, procarbazine, topotecan, nitrogen mustard, cytoxan, etoposide, 5-fluorouracil, bischloroethylnitrosourea, irinotecan, camptothecin, bleomycin, idarubicin, daunorubicin, dactinomycin, plicamycin, mitoxantrone, asparaginase, vinorelbine, chlorambucil, melphalan, carmustine, lomustine, busulfan, treosulfan, decarbazine, etoposide, teniposide, topotecan, 9-aminocamptothecin, crisnatol, mitomycin C, trimetrexate, mycophenolic acid, tiazofurin, ribavirin, EICAR (5-ethynyl-1-beta-D-ribofuranosy limidazole-4-carboxamide), hydroxyurea, deferoxamine, floxuridine, doxifluridine, raltitrexed, cytarabine (ara C), cytosine arabinoside, fludarabine, tamoxifen, raloxifene, megestrol, goserelin, leuprolide acetate, flutamide, bicalutamide, EB1089. CB1093, KH1060, verteporfin, phthalocyanine, photosensitizer Pe4, demethoxy-hypocrellin A, interferon-α, interferon-γ, tumor necrosis factor, gemcitabine, velcade, revamid, thalamid, lovastatin, 1-methyl-4-phenylpyridinium ion, staurosporine, actinomycin D, dactinomycin, bleomycin A2, bleomycin B2, peplomycin, epirubicin, pirarubicin, zorubicin, mitoxantrone, verapamil, thapsigargin, nucleases, and bacterial or plant-derived toxins, but are not limited to these. The anti-inflammatory agents may include steroids that reduce inflammation or swelling by binding to glucocorticoid receptors, non-steroidal anti-inflammatory drugs (NSAIDs) that alleviate pain by counteracting cyclooxygenase (COX) that synthesizes prostaglandins which cause inflammation, or immune-specific anti-inflammatory agents (ImSAIDs) that alter the activation and trafficking of inflammatory cells, but are not limited to these. The immunodisease therapeutics may include azathioprine, chlorambucil, cyclophosphamide, cyclosporine, mycophenolate, azathioprine, or methotrexate, but are not limited to these.

In the particles according to the present invention, the size of the particles may be from 10 nm to 1 cm. For example, the size of the particles may be from 100 nm to 5,000 nm. Preferably, the size of the calsequestrin-based metal ion-responsive particles may be from 1,000 nm to 5.000 nm.

In the particles according to the present invention, the particles may not be assembled by temperature changes.

In the particles according to the present invention, the particles may exclude polymers.

In the particles according to the present invention, the bioactive substance bound to the calsequestrin may be expressed in host cells by an expression vector containing a coding sequence for the calsequestrin and the bioactive substance or formed by chemical conjugation of calsequestrin and the bioactive substance.

In the particles according to the present invention, the particles can be used for the purpose of delivering bioactive substances to a target subject in need. The target subject may include, but is not limited to, humans, dogs, chickens, pigs, cattle, sheep, guinea pigs, or monkeys.

In the particles according to the present invention, the particles can be used for the purpose of tissue regeneration.

In the particles according to the present invention, the stability of the bioactive substance may be increased in vivo and ex vivo compared to before particle formation. For example, the stability of the bioactive substance may be increased by at least 1.1 times, 1.5 times, 1.7 times, 2 times, or more compared to before particle formation.

In the particles according to the present invention, the in vivo half-life of the bioactive substance may be increased compared to before particle formation. For example, the in vivo half-life of the bioactive substance may be increased by at least 1.1 times, 1.5 times, 1.7 times, 2 times, or more compared to before particle formation.

In the particles according to the present invention, the in vivo effect duration of the bioactive substance may be increased. For example, the in vivo effect duration of the bioactive substance may be increased by at least 1.1 times, 1.5 times, 1.7 times, 2 times, or more compared to before particle formation.

In the particles according to the present invention, the particles may further include an amino acid sequence that binds to a substance present in specific tissues or specific cells in the body to enable selective delivery of the particles to specific sites or cells in the body.

### II. Pharmaceutical Compositions and Therapeutic Methods

### Pharmaceutical Compositions

The present invention aims to provide a pharmaceutical composition for the treatment or prevention of diseases, which includes particles comprising calsequestrin, a bioactive substance bound to the calsequestrin, and a metal ionically bonded to the calsequestrin. In this case, the bioactive substance bound to the calsequestrin includes all the information described in the section I.

In the pharmaceutical composition according to the present invention, the disease may be one or more selected from the group consisting of skin wounds, diabetes, inflammatory diseases, ophthalmic diseases, tissue regeneration, infectious diseases, and cancer, but is not limited thereto.

In the pharmaceutical composition according to the present invention, the inflammatory diseases may include, but are not limited to, endocarditis, pericarditis, myocarditis, stomatitis, hepatitis, cholecystitis, cholangitis, esophagitis, colitis, gastritis, enteritis, appendicitis, pancreatitis, bronchitis, thyroiditis, oophoritis, cystitis, urethritis, prostatitis, vaginitis, osteomyelitis, arthritis, and any other inflammatory diseases known in the art.

In the pharmaceutical composition according to the present invention, the cancer diseases may include, but are not limited to, liver cancer, thyroid cancer, ovarian cancer, multiple myeloma, lymphoma, leukemia, kidney cancer, gastric cancer, breast cancer, cervical cancer, prostate cancer, pancreatic cancer, lung cancer, and all other cancer diseases known in the art.

In the pharmaceutical composition according to the present invention, the ophthalmic diseases may include, but are not limited to, macular degeneration, choroidal neovascularization, acute macular neuroretinopathy, macular edema, Behçet's disease, retinal disorders, diabetic retinopathy, retinal artery occlusive disease, central retinal vein occlusion, uveitic retinal diseases, retinal detachment, ocular trauma affecting the posterior segment or location, posterior segment diseases induced or affected by ocular laser treatments, posterior segment diseases induced or affected by photodynamic therapy, photocoagulation, radiation retinopathy, retinal membrane disorders, retinal branch vein occlusion, anterior ischemic optic neuropathy, non-retinopathy diabetic retinal dysfunction, retinitis pigmentosa, glaucoma, as well as anophthalmia, microphthalmia, astigmatism, blepharospasm, cataracts, conjunctival diseases, conjunctivitis, corneal diseases, corneal ulcers, dry eye syndrome, eyelid disorders, lacrimal duct diseases, lacrimal duct obstruction, myopia, presbyopia, pupil disorders, refractive errors, and strabismus, among others, but not limited thereto, and includes all ophthalmic diseases known in the art.

In the pharmaceutical composition according to the present invention, the infectious diseases may include diseases or conditions caused by bacteria, viruses, fungi, or parasites. For example, the infectious diseases may include, but are not limited to, influenza virus, flavivirus, human adenovirus (HAdV), severe acute respiratory syndrome coronavirus (SARS-COV or SARS-CoV-1), severe acute respiratory syndrome coronavirus type 2 (SARS-COV-2), Middle East respiratory syndrome coronavirus (MERS-COV), coronavirus disease 2019 (COVID-19), herpes virus, Zika virus. Japanese encephalitis virus (JEV), Epstein-Barr virus (EBV), Ebola virus (EBOV), rhinovirus, Chikungunya virus (CHIKV), hepatitis C virus (HCV), hepatitis B virus (HBV), hepatitis A virus (HAV), rotavirus, astrovirus, hantavirus, dengue virus, severe fever with thrombocytopenia syndrome virus (SFTS virus), HIV virus, West Nile Virus (WNV), yellow fever virus, and all other infectious diseases known in the art.

In the pharmaceutical composition according to the present invention, the pharmaceutical composition may further include a carrier, wherein the carrier is a pharmaceutically acceptable carrier commonly used in formulations, such as lactose, dextrose, sucrose, sorbitol, mannitol, starch, gum arabic, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, and mineral oil, but is not limited thereto. One example of the pharmaceutical composition of the present invention may include additional excipients such as lubricants, wetting agents, sweeteners, flavoring agents, emulsifiers, suspending agents, preservatives, etc., beyond the aforementioned components. Suitable pharmaceutically acceptable carriers and formulations are described in detail in Remington's Pharmaceutical Sciences (19th ed., 1995).

In the pharmaceutical composition according to the present invention, the pharmaceutical composition may be manufactured by mixing one or more diluents or excipients, such as fillers, extenders, binders, wetting agents, disintegrants, and surfactants commonly used in the art. For example, solid dosage forms for oral administration may include tablets, pills, powders, granules, capsules, lozenges, etc., and these solid dosage forms may be prepared by mixing the particles of the present invention with one or more excipients such as starch, calcium carbonate, sucrose, or lactose or gelatin. Additionally, lubricants such as magnesium stearate and talc may be used alongside simple excipients. Liquid dosage forms for oral administration may include suspensions, solutions, emulsions, or syrups, which may contain various excipients such as wetting agents, sweeteners, flavoring agents, and preservatives in addition to common diluents like water and liquid paraffin.

Formulations for non-oral administration include sterile aqueous solutions, non-aqueous solvents, suspensions, emulsions, freeze-dried preparations, suppositories, etc. Non-aqueous solvents and suspensions may include propylene glycol, polyethylene glycol, plant oils such as olive oil, injectable esters like ethyl oleate, and others. Suppository bases may include Witepsol, macrogol, Tween 61, cocoa butter, laurin, glycerol, gelatin, and others.

### Therapeutic Methods

The present invention aims to provide a method for treating diseases by administering a pharmaceutical composition comprising particles that include calsequestrin, a bioactive substance bound to the calsequestrin, and a metal ionically bonded to the calsequestrin, to an individual in need of such treatment.

In the therapeutic method according to the present invention, the individual may include humans, dogs, chickens, pigs, cattle, sheep, guinea pigs, or monkeys, but is not limited to these. In the therapeutic method according to the present invention, the administration can be oral, rectal, transdermal, intradermal, intravenous, intramuscular, intraperitoneal, intravascular, intramedullary, subdural, or subcutaneous. The dosage will vary depending on the patient's condition and weight, the severity of the disease, the form of the drug, the route of administration, and the timing of administration, but can be appropriately selected by those skilled in the art.

In the therapeutic method according to the present invention, the pharmaceutical composition is administered in a pharmaceutically effective amount. The term “pharmaceutically effective amount” refers to a sufficient amount for treating the disease with a reasonable benefit/risk ratio applicable to medical treatment. The effective dose level can be determined based on factors such as the type and severity of the patient's disease, the activity of the drug, sensitivity to the drug, timing of administration, route of administration, elimination rate, treatment period, concurrent medications, and other well-known factors in the medical field. The composition can be administered either as a single treatment or in combination with other treatments, sequentially or simultaneously with conventional treatments, and can be administered once or multiple times. It is important to administer an amount that achieves maximum efficacy with minimal side effects, which can be easily determined by those skilled in the art.

Specifically, the effective amount of the composition according to the present invention may vary depending on the patient's age, sex, and weight, and is generally administered at a dosage of 0.1 to 100 mg per kilogram of body weight per day or every other day, preferably 0.5 to 10 mg daily or divided into 1 to 3 doses per day. However, the dosage may be adjusted based on the route of administration, severity of obesity, sex, weight, age, and other factors, so the specified dosage should not be construed as limiting the scope of the present invention.

### III. Vaccine

The present invention provides vaccines for the prevention or treatment of one or more diseases selected from the group consisting of inflammatory diseases, infectious diseases, and cancer. These vaccines include particles comprising calsequestrin, a bioactive substance bound to the calsequestrin, and a metal ionically bonded to the calsequestrin. The bioactive substance bound to the calsequestrin includes all content described in section I. Inflammatory diseases, infectious diseases, and cancer include all content described in section II.

In the vaccine according to the present invention, the vaccine may further include an adjuvant. The adjuvant may include mineral substances, aluminum hydroxide, aluminum phosphate, bacterial extracts (e.g., bacterial lipopolysaccharides, protein adjuvants, and/or MDP), oily emulsions, saponins, squalene, potassium aluminum sulfate, calcium hydroxide, TLR agonists, etc., but is not limited to these.

In the vaccine according to the present invention, the vaccine may include a pharmaceutically acceptable carrier. The carrier may include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oil, among others, but is not limited to these.

In the vaccine according to the present invention, the vaccine may be formulated for non-oral administration, including subcutaneous or intradermal injection.

In the vaccine according to the present invention, the vaccine may be freeze-dried.

### IV. Kit

The present invention aims to provide a kit for manufacturing particles comprising calsequestrin and a bioactive substance configured to be attached to the calsequestrin. The bioactive substance bound to the calsequestrin includes all content described in section I.

In the kit according to the present invention, the kit may further include a metal configured to ionically bind to the calsequestrin. The metal may be selected from the group consisting of Cu, Ra, Ba, Sr. Ca, Cd, Co, Cr, Be, Fe(II), Zn. Mg, Mn, Ni, Al, Fe(III). Au, He, Si. V, Ga, In, La, and Ce, among others, but is not limited to these.

In the kit according to the present invention, the metal ion may be in the form of a metal compound or its aqueous solution. For example, if the metal ion is calcium ion, it may be included in the form of calcium chloride (CaCl2) or a solution of calcium chloride (CaCl2).

In the kit according to the present invention, the kit may include diluents, buffers, enzymes, instructions for preparing metal ion-reactive particles, etc., but is not limited to these.

In the kit according to the present invention, the metal ion may be present in a concentration of 0.1 to 10.000 mM, preferably 1 mM to 5,000 mM, more preferably 2 mM to 10,000 mM, and most preferably 10 mM to 1,000 mM.

In the kit according to the present invention, the bioactive substance bound to the calsequestrin may be included in the form of a nucleotide molecule encoding it.

In the kit according to the present invention, the bioactive substance bound to the calsequestrin may be included in the form of an expression vector containing nucleic acids encoding it.

In the kit according to the present invention, the bioactive substance bound to the calsequestrin may be included in the form of a cell transformed with an expression vector containing nucleic acids encoding it.

In the kit according to the present invention, the bioactive substance bound to the calsequestrin may be included in the form of a protein expressed from a cell transformed with an expression vector containing nucleic acids encoding it.

In the kit according to the present invention, the bioactive substance bound to the calsequestrin may be included in the form of a chemically conjugated form of calsequestrin and the bioactive substance.

### V. Manufacturing Method

The present invention provides a method for manufacturing particles comprising calsequestrin, a bioactive substance bound to the calsequestrin, and a metal ionically bonded to the calsequestrin. The bioactive substance bound to the calsequestrin includes all content described in section I.

In the manufacturing method according to the present invention, the method may include the step of mixing the bioactive substance attached to calsequestrin with the metal.

In the manufacturing method according to the present invention, the method may further include the step of attaching calsequestrin to the bioactive substance.

In the manufacturing method according to the present invention, the method may further include the step of recombinantly expressing the sequence containing calsequestrin and the bioactive substance.

In the manufacturing method according to the present invention, the method may further include the step of purifying the bioactive substance bound to calsequestrin.

In the manufacturing method according to the present invention, the method may include:
A) Manufacturing an expression vector containing nucleic acids coding for the bioactive substance bound to calsequestrin;
B) Transforming the expression vector into a host cell to obtain a transformed cell;
C) Expressing the protein containing the bioactive substance bound to calsequestrin from the transformed cell; and
D) Using metal ions to form particles of the expressed protein containing the bioactive substance bound to calsequestrin.

In the manufacturing method according to the present invention, the host cell may be selected from the group consisting of Escherichia coli, Bacillus subtilis, Bacillus thuringiensis, Salmonella typhimurium, Serratia marcescens. Pseudomonas species, yeast, insect cells, CHO cell lines (Chinese hamster ovary), W138, BHK, COS-7, 293. HepG2, 3T3, RIN, MDCK cell lines, and plant cells, among others.

In the manufacturing method according to the present invention, the method may include:
A) Reacting calsequestrin and the bioactive substance to form a chemical conjugation; and
B) Using metal ions to form particles of the calsequestrin-bound bioactive substance.

In the manufacturing method according to the present invention, the method may further include preparing the bioactive substance to form a chemical conjugation with calsequestrin.

### VI. Delivery Method

The present invention aims to provide a method for delivering bioactive substances to an individual in need. The bioactive substance bound to the calsequestrin includes all content described in section I.

The method for delivering bioactive substances according to the present invention comprises the steps of preparing particles according to the method described in section V, and administering the particles to a subject. The method for delivering bioactive substances according to the present invention may include administering calsequestrin, a bioactive substance, and a metal to a subject to form particles as described in section I. In the method for delivering bioactive substances according to the present invention, the calsequestrin can be linked to the bioactive substance. The method may also involve expressing nucleotide molecules encoding calsequestrin and the bioactive substance in the subject. Alternatively, the administration may involve expressing a first nucleotide molecule encoding calsequestrin and a second nucleotide molecule encoding the bioactive substance in the subject. The method for delivering bioactive substances according to the present invention can include various routes of administration, such as parenteral, oral, subcutaneous, topical, intramuscular, transdermal, buccal, sublingual, intranasal, intravascular, suborbital, or respiratory administration.

In the method for delivering bioactive substances according to the present invention, the bioactive substance can be administered directly to the target site of a disease in the subject. Alternatively, the bioactive substance may not be administered directly to the target site of the disease in the subject. The method may further include the step of releasing the bioactive substance from the particles. This release can occur either as the free form of the bioactive substance or as the bioactive substance linked to calsequestrin.

The present invention also seeks to provide the use of a product or method characterized by one or more elements disclosed in this application.

### Example Embodiment of the Invention

The following describes the present invention in more detail through examples. These examples are intended only to illustrate the present invention, and it will be apparent to those skilled in the art that the scope of the present invention is not construed as being limited by these examples.

### [1. Manufacture and Characterization of Metal Ion Particulated CSQ-KGF]

### Manufacturing Example 1. Manufacture of Metal Ion Particulated CSQ-KGF

### 1-1. Cloning of CSQ-KGF

In order to clone KGF into a CSQ-MCS vector, KGF-1 gene was synthesized (Bioneer, Daejeon, Korea). At this time, amino acid sequence information of CSQ and KGF-1 is as follows:

Human CSQ1 (SEQ ID No. 1):
QEGLDFPEYDGVDRVINVNAKNYKNVFKKYEVLALLYHEPPEDDKASQR
QFEMEELILELAAQVLEDKGVGFGLVDSEKDAAVAKKLGLTEVDSMYVF
KGDEVIEYDGEFSADTIVEFLLDVLEDPVELIEGERELQAFENIEDEIK
LIGYFKSKDSEHYKAFEDAAEEFHPYIPFFATFDSKVAKKLTLKLNEID
FYEAFMEEPVTIPDKPNSEEEIVNFVEEHRRSTLRKLKPESMYETWEDD
MDGIHIVAFAEEADPDGFEFLETLKAVAQDNTENPDLSIIWIDPDDFPL
LVPYWEKTFDIDLSAPQIGVVNVTDADSVWMEMDDEEDLPSAEELEDWL
EDVLEGEINTEDDDDDDDD
KGF (SEQ ID No. 12):
MCNDMTPEQMATNVNCSSPERHTRSYDYMEGGDIRVRRLFCRTQWYLRI
DKRGKVKGTQEMKNNYNIMEIRTVAVGIVAIKGVESEFYLAMNKEGKLY
AKKECNEDCNFKELILENHYNTYASAKWTHNGGEMFVALNQKGIPVRGK
KTKKEQKTAHFLPMAIT

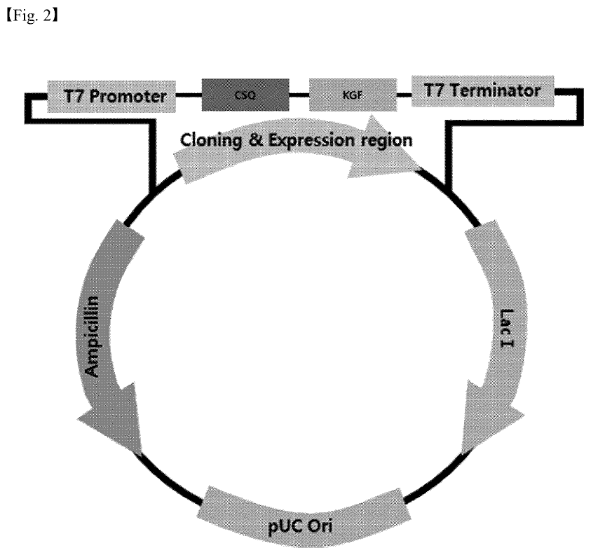

In order to insert the prepared KGF gene into the CSQ vector, the CSQ vector and insert DNA were treated with restriction enzymes. About 1 μg of the insert DNA was incubated overnight with BamHI (New England Biolabs (NEB, Ipswich) and XhoI (NEB, Ipswich), followed by purification of DNA using a PCR purification kit. In addition, about 40 μg of the CSQ vector was incubated for 3 hours with CIAP (Calf Intestinal Alkaline Phosphatase) (NEB, Ipswich), followed by purification with a PCR purification kit. The insert DNA was ligated to the CSQ vector at room temperature for 3 hours in the presence of T4 DNA ligase (Bioneer Corporation, Daejeon, Korea) (FIG. 2). Next, transformation was made of the DNA resulting from ligating the KGF insert to the CSQ vector. After being thawed on ice, 100 μl of DH5a competent cells was mixed and reacted for 30 min with 2 μl of the ligate solution. Heat shock was performed at 42° C. for 1 min on the reaction mixture which was then added with 200 μl of SOC medium and cultured at 37° C. for 30 minutes before being spread on plates. Of the colonies thus formed on the plate, six colonies were randomly picked up. The inserts were identified by PCR and DNA electrophoresis. The cloned colonies were committed to sequencing in Bioneer Corporation.

### 1-2. Purification of CSQ-KGF

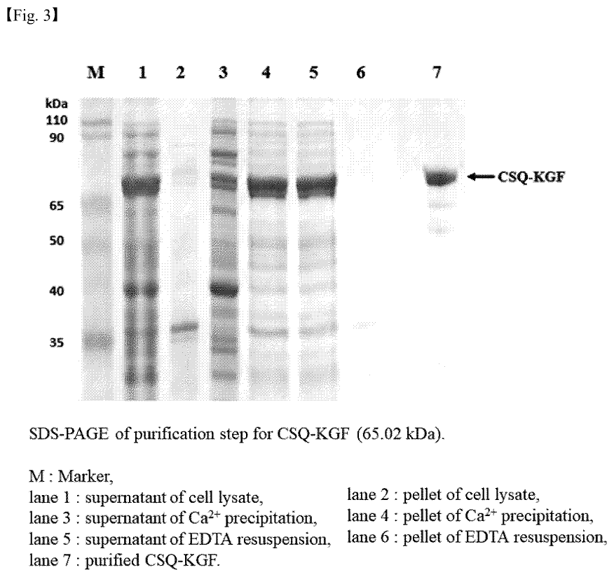

The DNAs sequenced in Bioneer Corporation were all transformed into BL21 cells which were then spread on agar plates containing ampicillin. Colonies grown on the agar plates were each inoculated into 5 ml of LB broth containing ampicillin (50 μg/ [symbol] ) and cultured overnight at 37° C. while stirring at 200 rpm. The cell culture was mixed at a ratio of 1:1 with glycerol to give a 1-ml stock and stored at −80° C. in a deep-freezer. The stock was inoculated in an amount of 20 μl into 20 ml of LB broth containing ampicillin (50 μg/ [symbol] ) and cultured overnight at 37° C. while stirring at 150 rpm. The culture was transferred to 400 ml of LB broth containing ampicillin (50 μg/ [symbol] ) and incubated until OD=1.2. Then, additional incubation was carried out overnight at 20° C. in the presence of 1 mM isopropyl-β-D-thiogalatopyranoside (IPTG) while stirring at 150 rpm. After centrifugation at 4° C. and 4000 rpm for 20 minutes, the supernatant was discarded and the cell pellet was suspended in a lysis buffer (20 mM Tris (pH 8.0). The suspension was stored overnight at −80° C. and then completely thawed, followed by adding a solution of 10 mg of PMSF (phenyl methane sulfonyl fluoride) in 1 ml of DMSO to 1 ml of the thawed E, coli. The E, coli was lysed using a sonicator, followed by centrifugation at 4° C. and 13,000 rpm for 1 hour. The supernatant was incubated with 20 mM CaCl₂) at 4° C. for 1 hour and then centrifuged at 5000 rpm for 30 minutes. After removal of the supernatant, the pellet thus formed was suspended in EDTA to obtain CSQ-KGF proteins (FIG. 3).

### 1-3. Particulation of CSQ-KGF

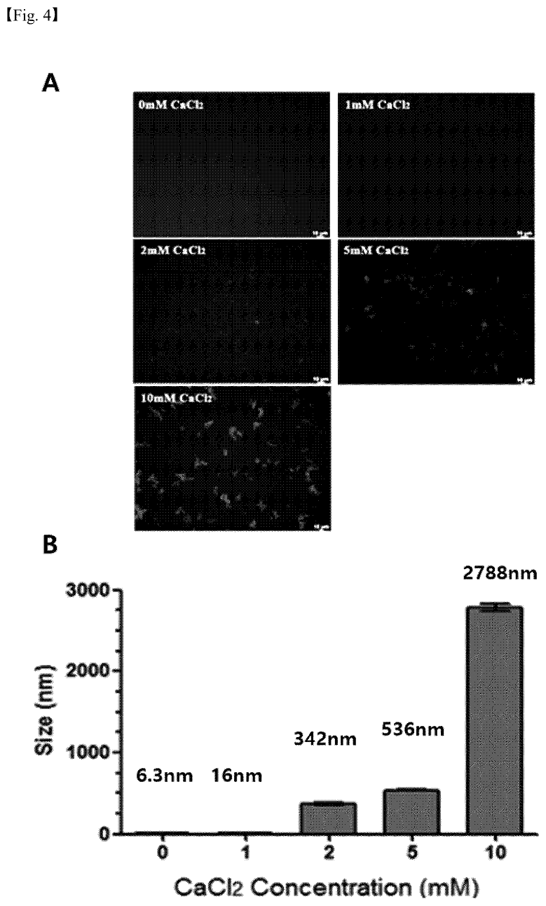

To assess whether the CSQ-KGF protein reacts with CaCl₂) and becomes stably particulate, CaCl₂ at concentrations of 1 mM to 10 mM was added to CSQ-KGF. After the reaction at 4° C. for 30 minutes, the particle size generated was measured through a fluorescence microscope and a dynamic light scattering photometer. As a result, it was observed that the CSQ-KGF protein can be stably particulated by reacting with metal ions, especially by metal ions at a concentration of 10 mM (FIG. 4).

### Example 1. Evaluation of the Characteristics of CSQ-KGF Particles

### 1-1. Stability Evaluation

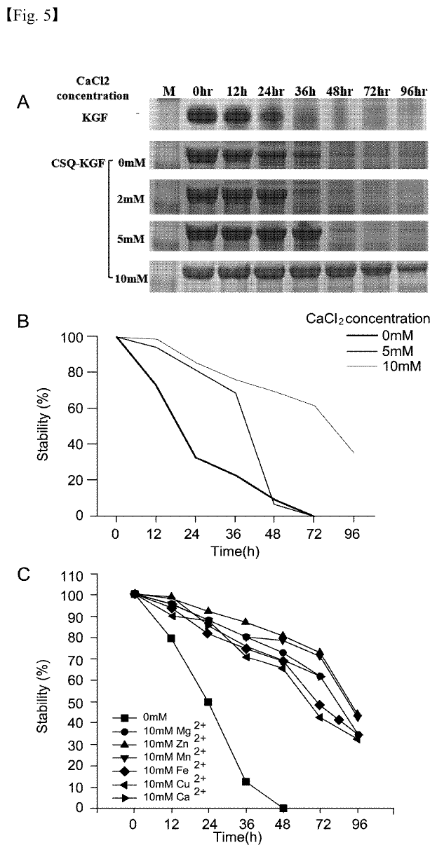

To 50 [symbol] of the purified CSQ-KGF protein, Ca2+, Mg2+, Fe2+, Zn2+, Cu2+, and Mn2+ were each added to a final concentration of 10 mM, and the mixture was incubated at 4° C. for 30 minutes to form particles. The medium in which the CHO cell was cultured was centrifuged at 13,000 rpm for 10 minutes to discard the supernatant. 50 [symbol] of the supernatant of the culture medium containing hydrolase was added to the particulated CSQ-KGF, lightly inverted 10 times, mixed, and then stored in an incubator at 37° C. for 3 days. After storage, samples were taken out at 12 or 24 hours intervals from 0 to 96 hours and stored at −20° C. After 3 days, the sample was completely thawed. 12.5 [symbol] of 5× sample buffer was added, and completely mixed by vortexing. The sample was heated at 80° C. for 10 minutes and then aliquoted into a well of 10% acrylamide SDS-PAGE gel in 10 [symbol] to check the protein band. Then, the SDS-PAGE gel was stained with a comassic brilliant blue and photographed by gel-documentation. Based on the band size of the photographed image, the stability of CSQ-KGF particulated with each metal ion was compared. As a result, when CSQ-KGF proteins are microsized by metal ions (Ca2+, Mg2+, Fe2+, Zn2+, Cu2+, and Mn2+), their stability is significantly improved compared to non-particulated CSQ-KGF (0) mM) (FIG. 5).

### 1-2. Evaluation of Emission Rate

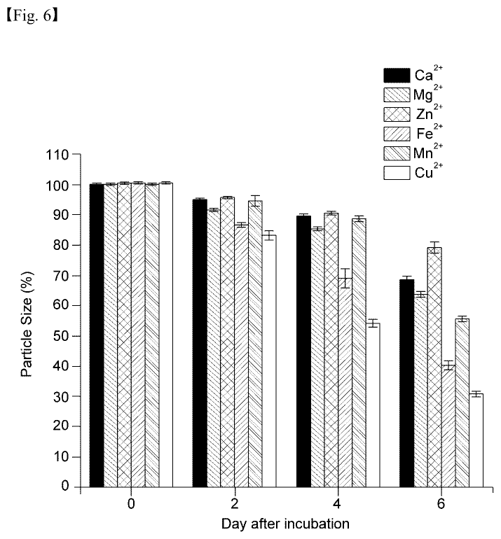

Ca2+, Mg2+, Fe2+, Zn2+, Cu2+, and Mn2+ ions were each added at a concentration of 10 mM to the purified CSQ-KGF, and the mixture was incubated at 4° C. for 30 minutes to form CSQ-KGF particles. The inlet of the tube containing the sample was sealed with parafilm and reacted at 37° C., and the particle size was measured with a dynamic light scattering meter every two days from 0 to 6 days to evaluate the emission rate. The inlet of the tube containing the sample was sealed with parafilm and incubated at 37° C. The particle size was measured using a dynamic light scattering meter every two days from day 0 to day 6 to evaluate the emission rate. As a result, it was observed that KGF is delayed when the CSQ-KGF protein is microsized by metal ions (Ca2+, Mg2+, Fe2+, Zn2+, Cu2+, and Mn2+) (FIG. 6).

### Example 1. Evaluation of the Efficacy of CSQ-KGF Particles

### 1-1. In Vitro Proliferation Test

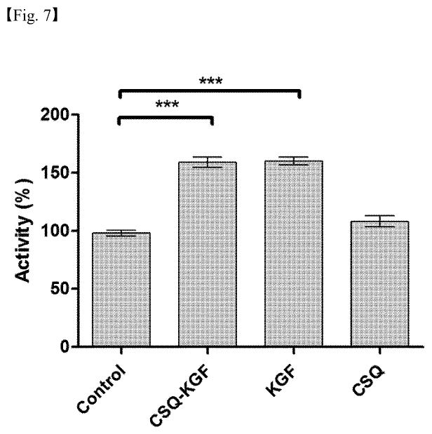

HaCaT cells in 96-well plates were aliquoted at a specific cell/well and incubated at 37° C. in a 5% CO2 incubator with 10% FBS and 1% P/S-DMEM for one day. Starvation was performed with FBS Free DMEM for one day. After washing twice with DPBS, CSQ-KGF, CSQ, and KGF were added to FBS-free DMEM at a concentration of 2.5 nM, and the sample was divided so that the total volume became 200 [symbol] . After incubation in an incubator for 2 days, XTT cell availability kit reagents were aliquoted, and then 450 nm absorbance was determined with a microplate reader. As a result, it was observed that KGF maintains the same efficacy as native KGF, even when fused with calsequestrin (CSQ-KGF) (FIG. 7).

### 1-2. In Vitro Migration Test

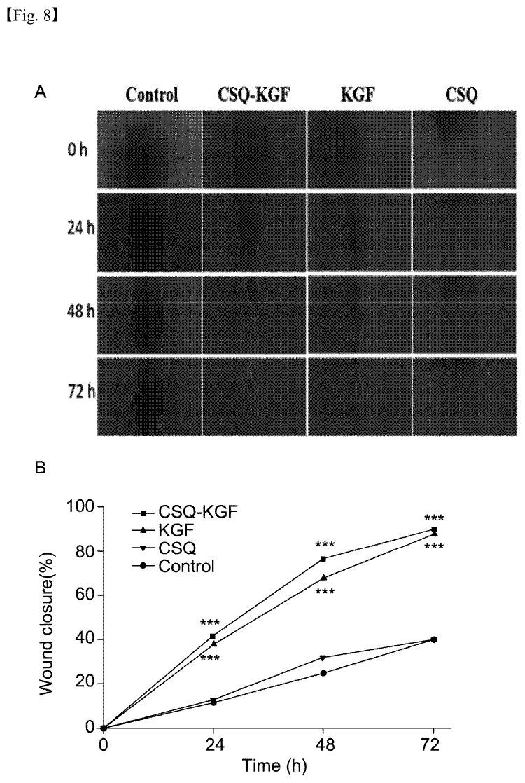

HaCaT cells were aliquoted into 24-well plates at a specific cell/well and incubated at 37° C. in a 5% CO2 incubator with 10% FBS and 1% P/S-DMEM for one day. Starvation was performed with FBS Free DMEM for one day. Cells were scratched in a straight line using a 1000 [symbol] pipette tip. After washing twice with DPBS, CSQ-KGF, CSQ, and KGF were added to FBS Free DMEM at a concentration of 10 nM, and the sample was divided so that the total volume became 500 [symbol] . While culturing in a 5% CO2 incubator at 37° C. scratches were observed using a phase-contrast microscope at 24-hour intervals from 0 to 74 hours, and photographs were taken. The extent of wound closure was compared by analyzing the scratch area of the captured images using ImageJ. As a result, it was observed that KGF maintains the same efficacy as native KGF, even when fused with calsequestrin (CSQ-KGF) (FIG. 8).

### 1-3. In Vivo Wound Recovery Rate Evaluation

C57BL/6N (Orient bio), female, and 8-week-old mouse were prepared in groups of 5 in a cage. During the 5-day adaptation period. 12-hr light-dark cycles were provided, and feed and drinking water were provided. Hair removal cream and clippers were used to remove hair on the back side.

One day after hair removal, four drosal full-thickness clouds were made per 1 using a 6 mm biopsy punch. CSQ-KGF microparticles (particulated with 10 mM CaCl₂)), non-particulated CSQ-KGF, KGF (in 20 mM Tris-HCl buffer, pH 7.0), and CSQ (in 20 mM Tris-HCl buffer, pH 7.0) were each prepared at a concentration of 1 nM, and 10 μl of each preparation was aliquoted onto the wound area. The control group was replaced with 20 mM Tris-HCl buffer (pH 7.0) 10 [symbol] . The wound was dressed with Tegadem. Pictures of the wound were taken at intervals of 2 days, and the length of the wound was measured using a digital caliper. The area of the wound was analyzed through imageJ, and the wound recovery rate was calculated according to Equation 1 below:

$$Wound recovery rate = ( initial wound area - open wound area ) / ( initial wound area ) \qquad Equation 1$$

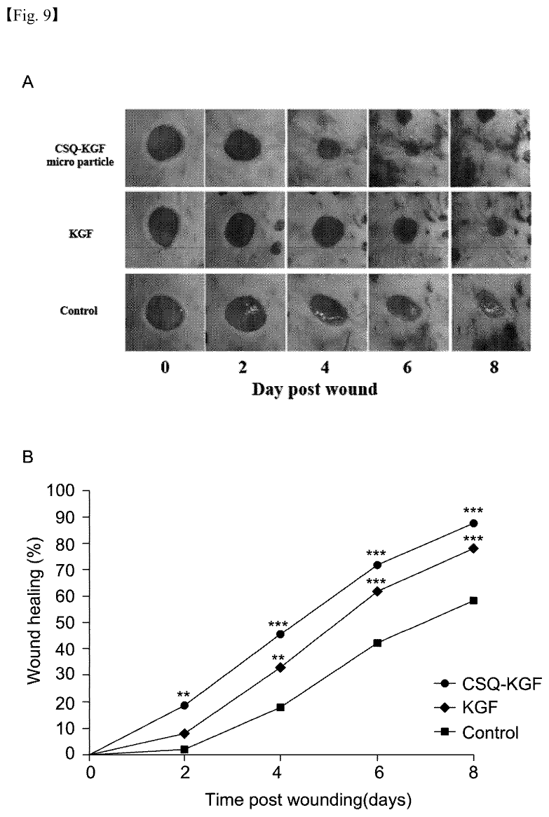

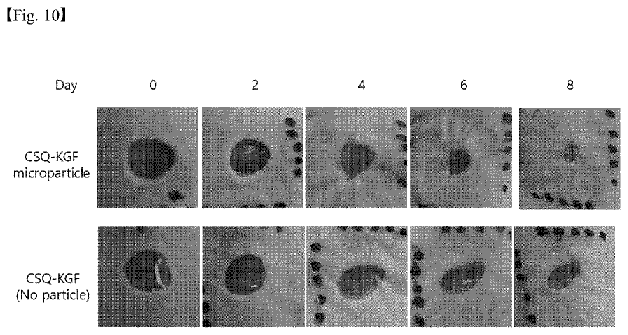

As a result, when CSQ-KGF protein is micro sized by metal ions, it has been confirmed that the wound recovery rate is significantly improved compared to control (Salin), native (KGF), and CSQ-KGF (No particle) proteins (FIG. 9-10).

### [2. Manufacturing and Characterization of Metal Ion Particulated Exenatide-CSQ]

### Manufacturing Example 2. Manufacture of Metal Ion Particulated Exenatide-CSQ

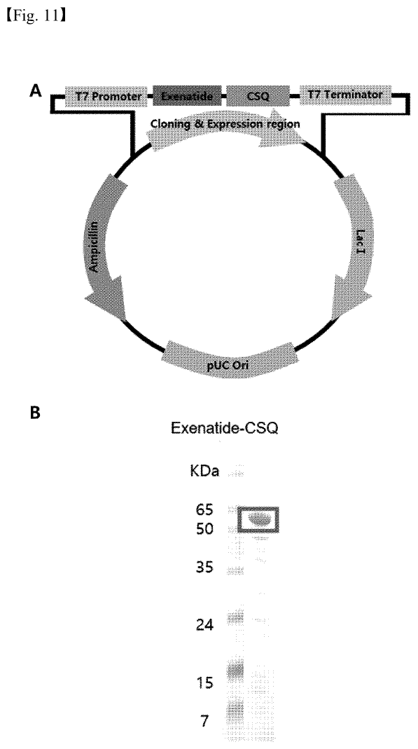

Exenatide-CSQ particles were prepared by using the Exenatide gene instead of the KGF-1 gene in the same manner as in Manufacturing Example 1 (FIG. 11). At this time, amino acid sequence information of Exenatide is as follows:

Exenatide (SEQ ID No. 13):
AAHGEGTFTSDLSKQMEEEAVRLFIEWLKNGGPSSGAPPPS

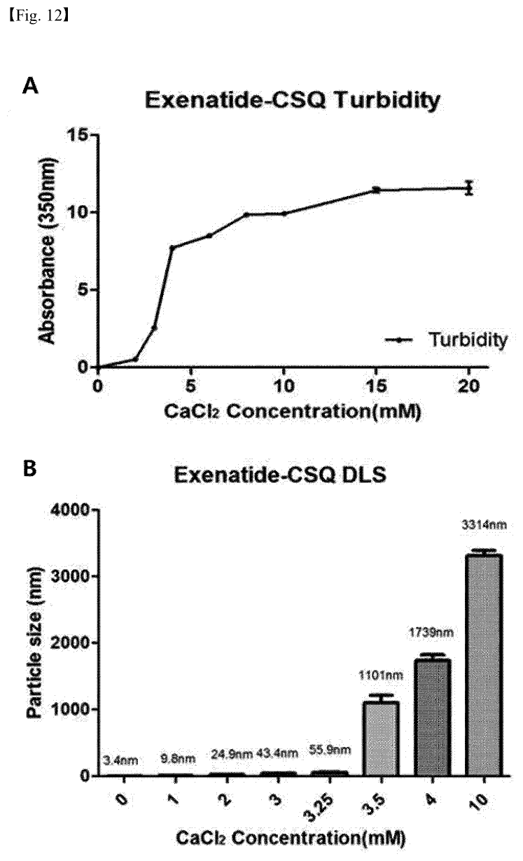

The size of the generated Exenatide-CSQ particles was measured using a fluorescence microscope and a dynamic light scattering photometer. As a result, it was observed that the Exenatide-CSQ protein could react with metal ions to stably form particles, especially at a concentration of 10 mM (FIG. 12).

### Example 2. Evaluation of the Characteristics of Exenatide-CSQ Particles

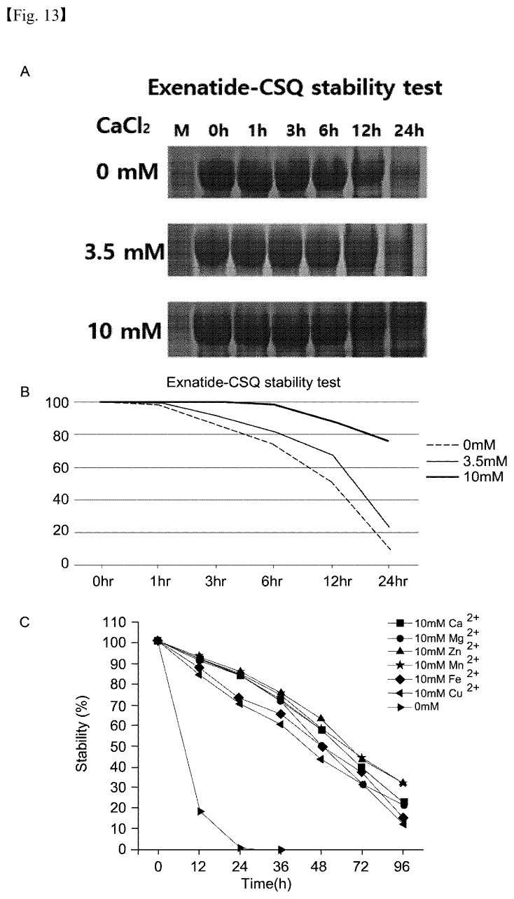

Ca2+, Mg2+, Fe2+, Zn2+, Cu2+, and Mn2+ ions were each added to 50 [symbol] of the purified Exenatide-CSQ protein and incubated at 4° C. for 30 minutes to form particles. The stability of the particulated Exenatide-CSQ with each metal ion was then compared using the same method as described in Example 1. As a result, when the Exenatide-CSQ protein is microsized by metal ions (Ca2+, Mg2+, Fe2+, Zn2+, Cu2+, and Mn2+), its stability is significantly improved compared to the non-particulated Exenatide-CSQ (0 mM) (FIG. 13).

### Example 2. Efficacy Evaluation of Exenatide-CSQ

### 2-1. In Vivo Glycemic Reduction Rate Assessment

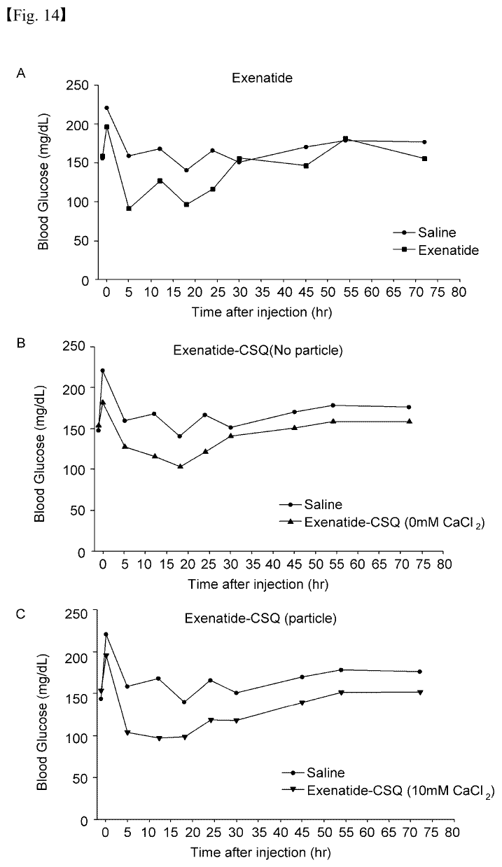

C57BL/6J (Orient bio), male, and 6-week-old mice were prepared in a cage in four groups of 4. During the 5-day adaptation period, 12-hour light-dark cycles were provided with feed and drinking water. Feed was stopped 20 hours before the experiment. Blood samples were collected via tail-cut 1 hour before drug injection, and blood glucose levels were measured using a Green Cross Green Doctor blood glucose meter. Exenatide, exenatide-CSQ (No particle), and exenatide-CSQ Depot (particle. 30 minutes reaction with 10 mM CaCl₂)) were injected by S.C Injection at a concentration of 750 nmol/kg, respectively. After injection, blood sugar was measured and the hypoglycemic effect was evaluated. As a result, it was observed that when Exenatide-CSQ protein is microsized by metal ions, it shows a significantly improved blood sugar reduction effect compared to native (Exenatide) and CSQ-KGF (No particle) that is not granulated by metal ions (FIG. 14).

### 2-2. In Vivo Half-Life Evaluation

After S.C. Injection of Exenatide-CSQ particles made of 10 mM calcium and Exenatide (No particle) and Exenatide, which are 0) mM calcium, into mice, serum samples were made according to time.

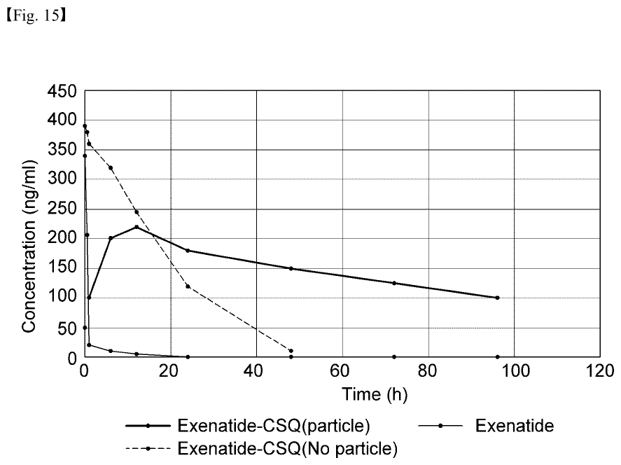

The half-life of each sample was measured using Exenatide ELISA kits (MyBioSource). As a result, it was observed that when Exenatide-CSQ proteins are microsized by metal ions, they can be maintained at high concentrations in vivo for a long time compared to native (Exenatide) and Exenatide-CSQ (No particles) that are not particulated by metal ions (FIG. 15).

### [3. Manufacturing and Characterization of Metal Ion Particulated CSQ-SARS Cov2 RBD]

### Manufacturing Example 3. Manufacture of Metal Ion Particulated CSQ-SARS Cov2 RBD

CSQ-SARS Cov2 RBD particles were prepared in the same method as in Manufacturing Example 1, except that the SARS Cov2 RBD gene was used instead of the KGF-1 gene. At this time, amino acid sequence information of SARS Cov2 RBD is as follows:

SARS Cov2 RBD (SEQ ID No. 14):
PNITNLCPFGEVFNATRFASVYAWNRKRISNCVADYSVLYNSASFSTFK
CYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAG
STPCNGVEGENCYFPLQSYGFQPTNGVGYQPYRVVVLSFELLHAPATVC
GPKKS

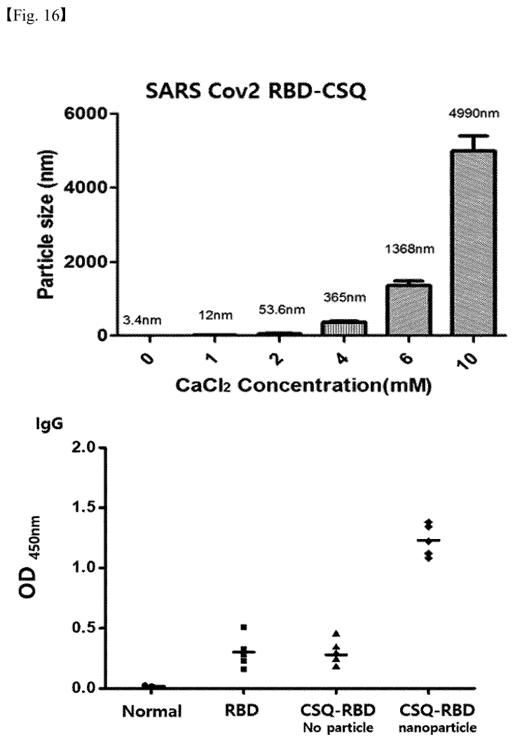

The size of the generated CSQ-SARS Cov2 RBD particles is measured using a fluorescence microscope and a dynamic light scattering method. As a result, it is observed that the CSQ-SARS Cov2 RBD protein reacts with metal ions and is stably particulated (FIG. 16A).

### Example 3. Immunogenicity Evaluation of CSQ-SARS Cov2 RBD

To confirm the immunogenicity of SARS Cov2 RBD-CSQ nanoparticles, 4 mM CaCl2 was treated to prepare 365 nm nanoparticles. Balb/c mice were prepared in groups of 5 in the cage. 50 μg of RBD-CSQ nanoparticles, RBD protein, and Saline were injected once, and then the second injection was performed 2 weeks later. On the 14th day after the second injection, blood samples were collected and analyzed by ELISA using an anti-RBD antibody. Briefly, 96-well plates (Cornings) were coated overnight with 100 [symbol] of antigen solution (30 μg/ml RBD). After the plates were washed and blocked, blood samples (a 1:10000 dilution in 2% milk) were added, and the plates were incubated for 1 hour at room temperature. The plates were then washed, HRP-conjugated anti-mouse IgG was used as a secondary antibody, TMB substrate (BD OptELA) was added, and absorbance was evaluated at 450 nm. As a result, when SARS Cov2 RBD-CSQ protein is particulated by metal ions, immunogenicity is significantly increased compared to native (SARS Cov2 RBD) and SARS Cov2 RBD-CSQ (No particle) that are not particulated by metal ions (FIG. 16B).

### [4. Manufacturing and Characterization of Metal Ion Particulated CSQ-Doxorubicin]

### Manufacturing Example 4. Manufacture of Metal Ion Particulated CSQ-Doxorubicin

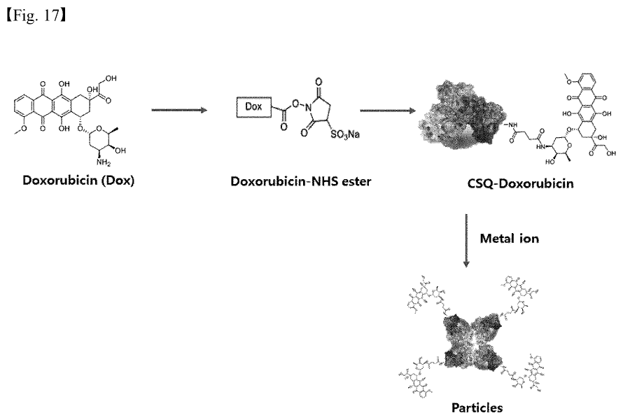

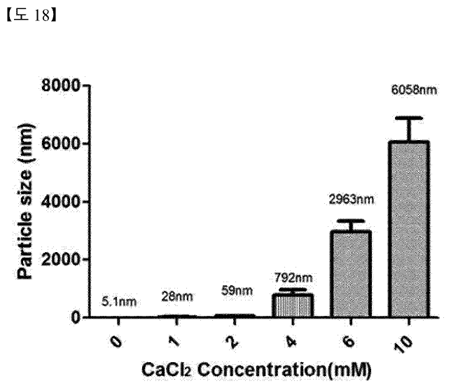

A doxorubicin conjugation kit (PerKit)) was purchased and doxorubicin was bound to CSQ according to the kit manual. Briefly, after making a doxorubicin-NHS ester by attaching a sulfo-NHS ester to doxorubicin, doxorubicin was mixed with 1 to 3 mg CSQ and reacted for 2 hours in a dark place. Then. Doxorubicin-CSQ and Doxorubicin were separated by Desalting column to obtain Doxorubicin-CSQ (FIG. 17). To evaluate the particulate production of the produced CSQ-Doxorubicin protein to CaCl₂), CaCl₂) at a concentration of 1 mM to 10 mM was added to CSQ-Doxorubicin. After reacting at 4° C. for 30 minutes, the particle size produced was measured using a dynamic light scattering photometer. As a result, it was observed that the CSQ-Doxorubicin protein reacts with metal ions and becomes stably particulated. (FIG. 18).

### Example 4: Assessment of the In Vivo Half-Life of CSQ-Doxorubicin

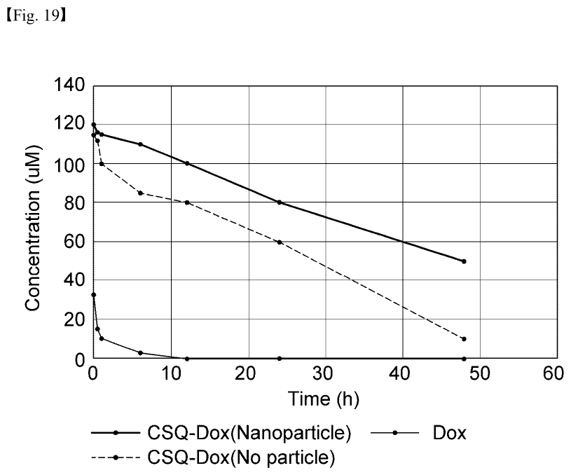

Doxorubicin-CSQ particles made of 2 mM calcium, Doxorubicin-CSQ particles (Dox-CSQ. No particles) and Doxorubicin (Dox), which are 0) mM calcium, were injected into BALB/c mice, and serum samples were made according to time. The amount of serum was measured using fluorescence of doxorubicin (Dox). As a result, it was observed that when CSQ-Doxorubicin protein is particulated by metal ions, it can be maintained at a high concentration in vivo for a long time compared to CSQ-Doxorubicin (No particle) that is not particulated by native (Doxorubicin) and metal ions (FIG. 19).

### [5. Manufacturing and Characterization of Metal Ion Particulated CSQ-EGF]

CSQ-EGF particles were prepared in the same method as described in Manufacturing Example 1, except that the EGF gene was used instead of the KGF-1 gene. In this case, the amino acid sequence information of EGF is as follows:

EGF (SEQ ID No. 15):
NSDSECPLSHDGYCLHDGVCMYIEALDKYACNCVVGYIGERCQYRDLKW
WELR

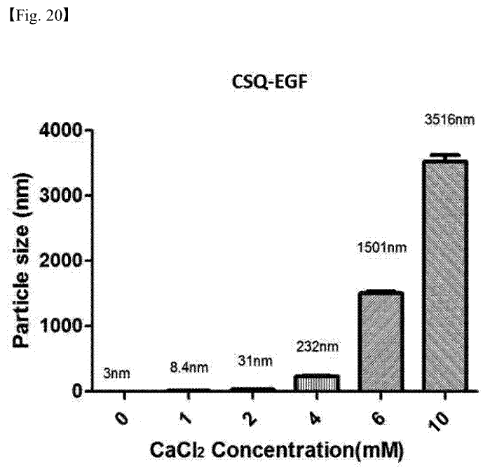

The size of the generated CSQ-EGF particles was measured using a fluorescence microscope and a dynamic light scattering photometer. As a result, it was observed that the CSQ-EGF protein reacts with metal ions and becomes stably particulate. (FIG. 20).

[6. Manufacture and Characterization of Metal Ion Particleized CSQ-Anti-VEGF scFv-CSQ]

CSQ-Anti-VEGF scFv-CSQ particles were prepared in the same method as described in Manufacturing Example 1, except that the anti-VEGF scFv-CSQ gene was used instead of the KGF-1 gene:

Anti-VEGF scFv (SEQ ID No. 16):
EIVMTQSPSTLSASVGDRVIITCQASEIIHSWLAWYQQKPGKAPKLLIY
LASTLASGVPSRFSGSGSGAEFTLTISSLQPDDFATYYCQNVYLASTNG
ANFGQGTKLTVLGGGGGSGGGGSGGGGSGGGGSEVQLVESGGGLVQPGG
SLRLSCTASGFSLTDYYYMTWVRQAPGKGLEWVGFIDPDDDPYYATWAK
GRFTISRDNSKNTLYLQMNSLRAEDTAVYYCAGGDHNSGWGLDIWGQGT
LVTVSS

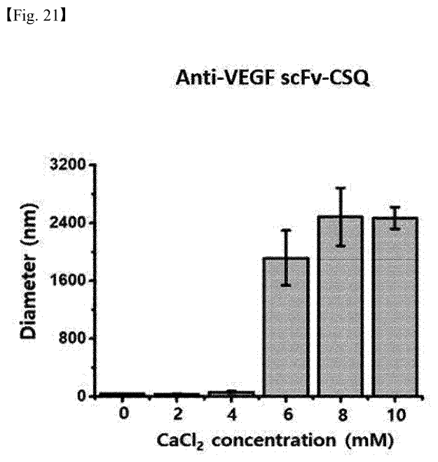

The size of the above generated Anti-VEGF scFv-CSQ particles was measured by fluorescence microscopy and dynamic light scattering photometry. As a result, it was confirmed that the CSQ-Anti-VEGF scFv-CSQ protein reacts with metal ions and stably particulates (FIG. 21).

### [7. Preparation and Characterization of Metal Ion Particulate CSQ-FGF2]

CSQ-FGF2 particles were prepared using the same method as in Example 1 above, except that the FGF2 gene was used instead of the KGF-1 gene. The amino acid sequence information of FGF2 is as follows:

FGF2 (Sequence #17):
PALPEDGGSGAFPPGHFKDPKRLYCKNGGFFLRIHPDGRVDGVREKSDP
HIKLQLQAEERGVVSIKGVCANRYLAMKEDGRLLASKCVTDECFFFERL
ESNNYNTYRSRKYTSWYVALKRTGQYKLGSKTGPGQKAILFLPMSAKS

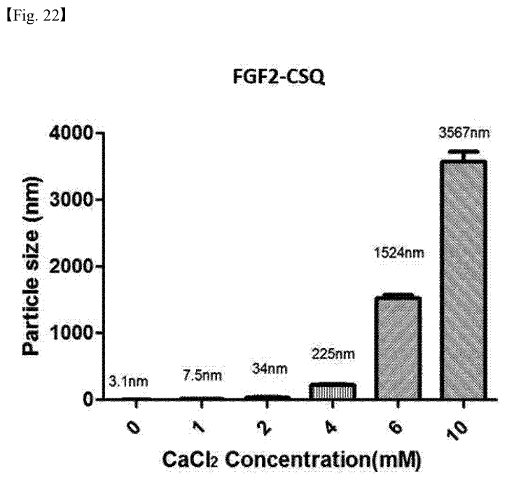

The size of the generated FGF2 particles was measured by fluorescence microscopy and dynamic light scattering. As a result, it was confirmed that the CSQ-FGF2 protein reacts with metal ions and stably particulates (FIG. 22).

### [8. Preparation and Characterization of Metal Ion Particulate CSQ-BMP2]

CSQ-BMP2 particles were prepared using the same method as in Example 1 above, except that the BMP2 gene was used instead of the KGF-1 gene. The amino acid sequence information of BMP2 is as follows:

BMP2 (Sequence #18):
MQAKHKQRKRLKSSCKRHPLYVDFSDVGWNDWIVAPPGYHAFYCHGECP
FPLADHLNSTNHAIVQTLVNSVNSKIPKACCVPTELSAISMLYLDENEK
VVLKNYQDMVVEGCGCR

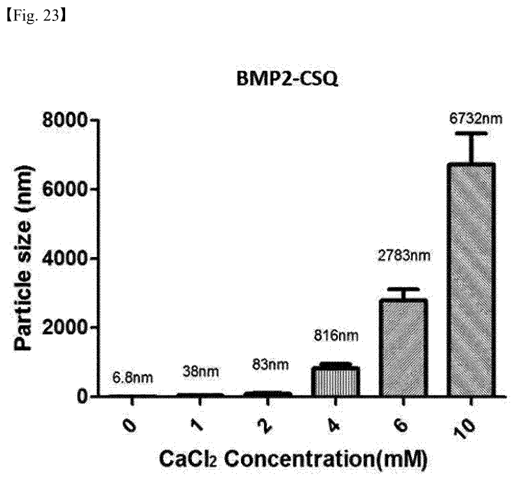

The size of the CSQ-BMP2 particles generated above was measured by fluorescence microscopy and dynamic light scattering photometry. As a result, it was confirmed that the CSQ-BMP2 protein reacts with metal ions and stably particulates (FIG. 23).

### [9. Preparation and Characterization of Metal Ion Particulate CSQ-TGF].

CSQ-TGF particles were prepared using the same method as in Example 1 above, except that the TGF gene was used instead of the KGF-1 gene. The amino acid sequence information of TGF is as follows:

TGF (Sequence #19):
ALDTNYCFSSTEKNCCVRQLYIDFRKDLGWKWIHEPKGYHANFCLGPCP
YIWSLDTQYSKVLALYNQHNPGASAAPCCVPQALEPLPIVYYVGRKPKV
EQLSNMIVRSCKCSR

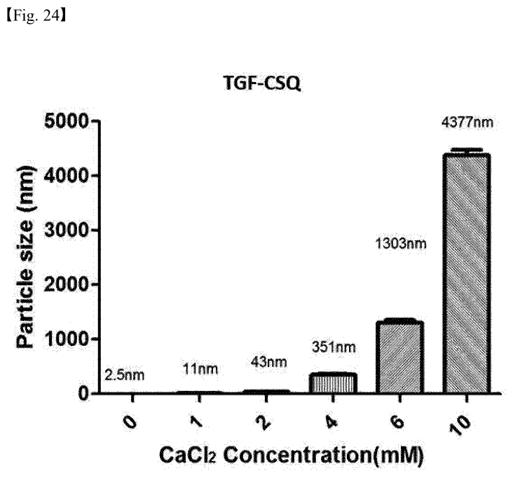

The size of the CSQ-TGF particles generated above was measured using fluorescence microscopy and dynamic light scattering photometry. The results confirmed that the CSQ-TGF protein reacts with metal ions to form stable particles (FIG. 24).

### [10. Preparation and Characterisation of Metal Ion Particulate CSQ-VEGF].

CSQ-VEGF particles were prepared using the same method as in Example 1 above, except that the VEGF gene was used instead of the KGF-1 gene.

VEGF (Sequence #20):
MNFLLSWVHWSLALLLYLHHAKWSQAAPMAEGGGQNHHEVVKFMDVYQR
SYCHPIETLVDIFQEYPDEIEYIFKPSCVPLMRCGGCCNDEGLECVPTE
ESNITMQIMRIKPHQGQHIGEMSFLQHNKCECRPKKDRARQEKCDKPRR

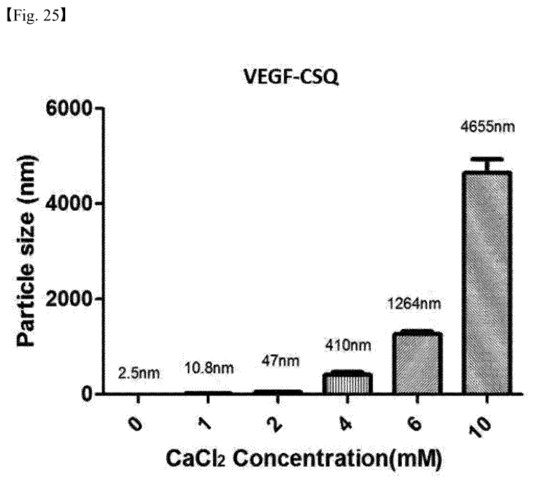

The size of the CSQ-VEGF particles generated above was measured by fluorescence microscopy and dynamic light scattering photometry. The results confirmed that the CSQ-VEGF protein reacts with metal ions to stably particulate (FIG. 25).

### [11. Preparation and Characterisation of Metal Ion Particulate CSQ-GLP1].

CSQ-GLP1 particles were prepared using the same method as in Example 1 above, except that the GLP1 gene was used instead of the KGF-1 gene.

GLP1 (Sequence #21):
AAHGEGTFTSDVSSYLEEQAAKEFIAWLVKGA

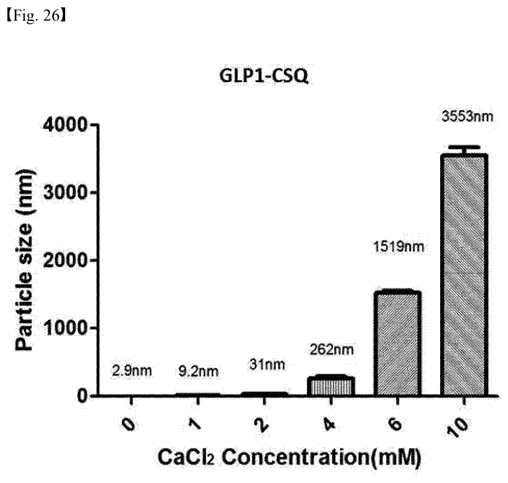

The size of the CSQ-GLP1 particles generated above was measured by fluorescence microscopy and dynamic light scattering photometry. The results confirmed that the CSQ-GLP1 protein reacts with metal ions and stably particulates (FIG. 26).

### [12. Preparation and Characterisation of Metal Ion Particulate CSQ-TEV Protease].

CSQ-TEV protease particles were prepared using the same method as described in Example 1 above, except that the TEV protease gene was used instead of the KGF-1 gene. The amino acid sequence information of TEV protease is as follows:

TEV protease (Sequence #22)
GESLFKGPRDYNPISSTICHLTNESDGHTTSLYGIGFGPFIITNKHLFR
RNNGTLLVQSLHGVFKVKNTTTLQQHLIDGRDMIIIRMPKDFPPFPQKL
KFREPQREERICLVTTNFQTKSMSSMVSDTSCTFPSSDGIFWKHWIQTK
DGQCGSPLVSTRDGFIVGIHSASNFTNTNNYFTSVPKNFMELLTNQEAQ
QWVSGWRLNADSVLWGGHKVFMVKPEEPFQPVKEATQLMN

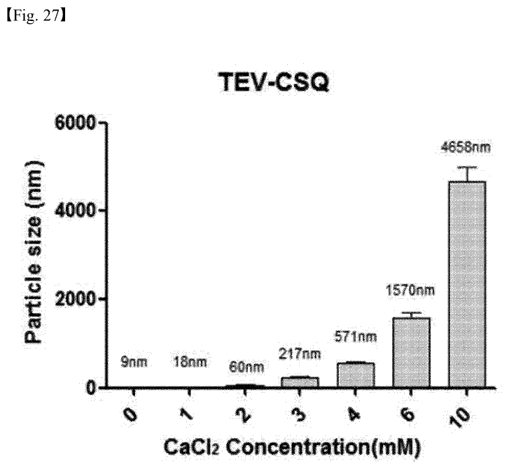

The size of the CSQ-TEV protease particles generated above was measured by fluorescence microscopy and dynamic light scattering photometry. As a result, it was confirmed that the CSQ-TEV protease protein reacts with metal ions and stably particulates (FIG. 27).

### [13. Preparation and Characterization of Metal Ion Particulate CSQ-HRP Enzyme].

### Example 13. Preparation of Metal Ionized CSQ-HRP Enzyme Particles

The CSQ-HRP enzyme particles were prepared using the same method as in Example 1 above, except that the HRP enzyme gene was used instead of the KGF-1 gene. The amino acid sequence information of HRP is shown below.

HRP (Sequence #23):
QLTPTFYDNSCPNVSNIVRDTIVNELRSDPRIAASILRLHFHDCFVNGC
DASILLDNTTSFRTEKDAFGNANSARGFPVIDRMKAAVESACPRTVSCA
DLLTIAAQQSVTLAGGPSWRVPLGRRDSLQAFLDLANANLPAPFFTLPQ
LKDSFRNVGLNRSSDLVALSGGHTFGKNQCRFIMDRLYNFSNTGLPDPT
LNTTYLQTLRGLCPLNGNLSALVDFDLRTPTIFDNKYYVNLEEQKGLIQ
SDQELFSSPNATDTIPLVRSFANSTQTFFNAFVEAMDRMGNITPLTGTQ
GQIRLNCRVVNSNSLLHDMVEVVDFVSSM

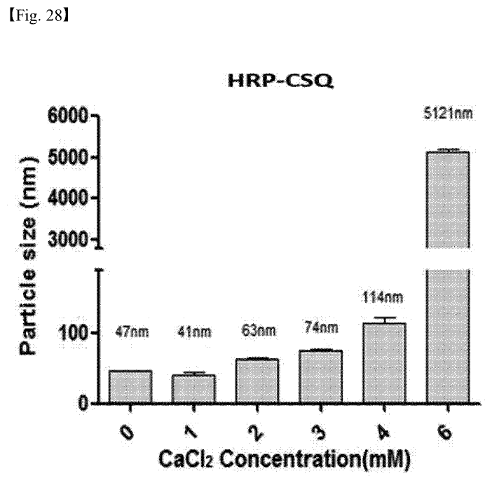

The size of the CSQ-HRP enzyme particles generated above was measured by fluorescence microscopy and dynamic light scattering photometry. As a result, it was confirmed that the CSQ-HRP enzyme protein reacts with metal ions and stably particulates (FIG. 28).

### Example 13. Stability Evaluation of HRP-CSQ Particles

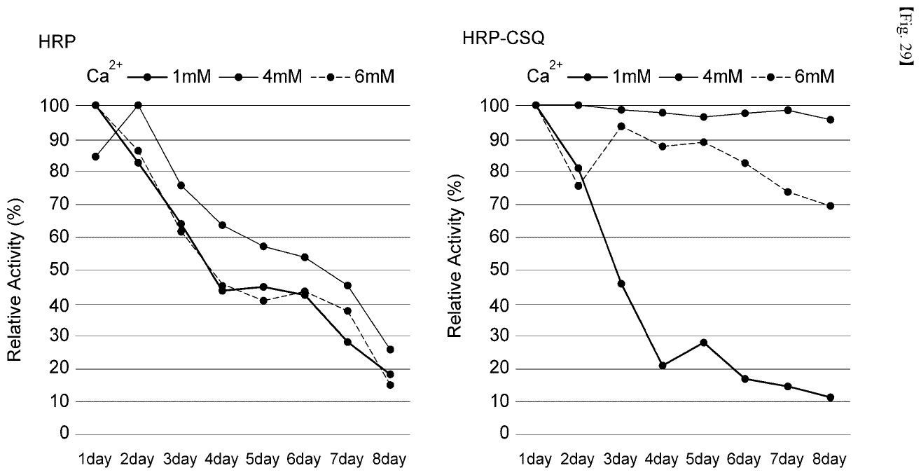

The HRP-CSQ protein was treated with 1 mM, 4 mM, and 6 mM of calcium in order to facilitate its particulate formation. Additionally, HRP protein was incorporated at 1 mM, 4 mM, and 6 mM of calcium as a control element. The two proteins were stored at 30° C. for eight days in order to measure the activity of HRP. The activity of HRP was quantified using a TMB solution. The results demonstrated that the stability of the HRP-CSQ protein, when particulate with metal ions, was enhanced in comparison to the un-particulated HRP-CSQ (0 mM). Notably, the activity remained stable for up to eight days (FIG. 29).

### [14. Preparation and Characterization of Metal Ion Particulate CSQ-hGH].

The CSQ-hGH particles were prepared using the same method as in Example 1, with the exception of the hGH gene, which was used in its place of the KGF-1 gene. The amino acid sequence information for hGH is as follows:

hGH (Sequence #24):
FPTIPLSRLFDNAMLRAHRLHQLAFDTYQEFEEAYIPKEQKYSFLQNPQ
TSLCFSESIPTPSNREETQQKSNLELLRISLLLIQSWLEPVQFLRSVFA
NSLVYGASDSNVYDLLKDLEEGIQTLMGRLEDGSPRTGQIFKQTYSKFD
TNSHNDDALLKNYGLLYCFRKDMDKVETFLRIVQCRSVEGSCGF

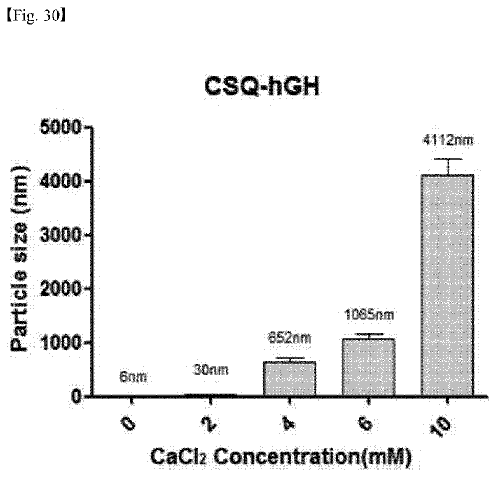

The size of the CSQ-hGH particles generated above was measured by fluorescence microscopy and dynamic light scattering photometry. As a result, it was confirmed that the CSQ-hGH protein reacts with metal ions and stably particulates (FIG. 30).

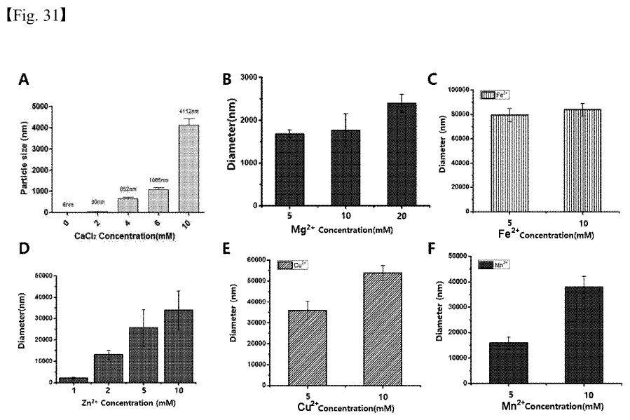

In addition, metal ions were added to CSQ-hGH at concentrations ranging from 1 mM to 10 mM to evaluate particulate generation with Ca2+, Mg2+, Fe2+, Zn2+, Cu2+, and Mn2+ ions in the CSQ-hGH protein. After a 30-minute reaction at room temperature, the resulting particle size was measured by dynamic light scattering photometry. The results confirmed that the CSQ-hGH protein reacts with various metal ions (Ca2+, Mg2+, Fe2+, Zn2+, Cu2+, Mn2+) and stably forms particulates (FIG. 31).

### [15. Preparation and Characterization of Metal Ion Particulate CSQ-GCSF].

CSQ-GCSF particles were prepared using the same method as described in Example 1 above, except that the GCSF gene was used instead of the KGF-1 gene. The amino acid sequence information of GCSF is as follows:

G-CSF (Sequence #25):
TPLGPASSLPQSFLLKCLEQVRKIQGDGAALQEKLCATYKLCHPEELVL
LGHSLGIPWAPLSSCPSQALQLAGCLSQLHSGLFLYQGLLQALEGISPE
LGPTLDTLQLDVADFATTIWQQMEELGMAPALQPTQGAMPAFASAFQRR
AGGVLVASHLQSFLEVSYRVLRHLAQP

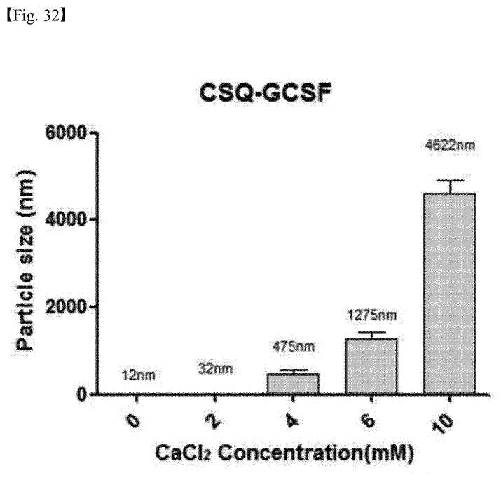

The size of the CSQ-GCSF particles generated above was measured by fluorescence microscopy and dynamic light scattering photometry. As a result, it was confirmed that the CSQ-GCSF protein reacts with metal ions and stably particulates (FIG. 32).

### [16. Preparation and Characterization of Metal Ion Particulate KGF-CSQ2(WT), KGF-CSQ2(Δ8), and KGF-CSQ2(Δ16)]

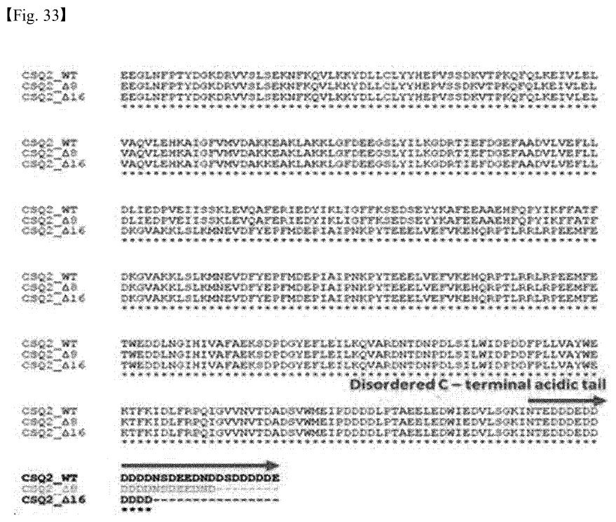

KGF-CSQ2(WT), KGF-CSQ2(Δ8), and KGF-CSQ2(Δ16) particles were prepared using the same method as described in Example 1 above, except that CSQ2(WT), CSQ2(Δ8), and CSQ2(Δ16) were used instead of CSQ (sequence number 1) (FIG. 33). The amino acid sequence information of CSQ2(WT), CSQ2(Δ8), and CSQ2(Δ16) is as follows:

Human CSQ2 (hCSQ2, Sequence #2):
EEGLNFPTYDGKDRVVSLSEKNFKQVLKKYDLLCLYYHEPVSSDKVTPK
QFQLKEIVLELVAQVLEHKAIGFVMVDAKKEAKLAKKLGFDEEGSLYIL
KGDRTIEFDGEFAADVLVEFLLDLIEDPVEIISSKLEVQAFERIEDYIK
LIGFFKSEDSEYYKAFEEAAEHFQPYIKFFATFDKGVAKKLSLKMNEVD
FYEPFMDEPIAIPNKPYTEEELVEFVKEHQRPTLRRLRPEEMFETWEDD
INGIHIVAFAEKSDPDGYEFLEILKQVARDNTDNPDLSILWIDPDDFPL
LVAYWEKTFKIDLFRPQIGVVNVTDADSVWMEIPDDDDLPTAEELEDWI
EDVLSGKINTEDDDEDDDDDDNSDEEDNDDSDDDDDE
CSQ2 Δ8 (Sequence #3):
EEGLNFPTYDGKDRVVSLSEKNFKQVLKKYDLLCLYYHEPVSSDKVTPK
QFQLKEIVLELVAQVLEHKAIGFVMVDAKKEAKLAKKLGFDEEGSLYIL
KGDRTIEFDGEFAADVLVEFLLDLIEDPVEIISSKLEVQAFERIEDYIK
LIGFFKSEDSEYYKAFEEAAEHFQPYIKFFATFDKGVAKKLSLKMNEVD
FYEPFMDEPIAIPNKPYTEEELVEFVKEHQRPTLRRLRPEEMFETWEDD
INGIHIVAFAEKSDPDGYEFLEILKQVARDNTDNPDLSILWIDPDDFPL
LVAYWEKTFKIDLFRPQIGVVNVTDADSVWMEIPDDDDLPTAEELEDWI
EDVLSGKINTEDDDEDDDDDDNSDEEDND
CSQ2 Δ16 (Sequence #4):
EEGLNFPTYDGKDRVVSLSEKNFKQVLKKYDLLCLYYHEPVSSDKVTPK
QFQLKEIVLELVAQVLEHKAIGFVMVDAKKEAKLAKKLGFDEEGSLYIL
KGDRTIEFDGEFAADVLVEFLLDLIEDPVEIISSKLEVQAFERIEDYIK
LIGFFKSEDSEYYKAFEEAAEHFQPYIKFFATFDKGVAKKLSLKMNEVD
FYEPFMDEPIAIPNKPYTEEELVEFVKEHQRPTLRRLRPEEMFETWEDD
INGIHIVAFAEKSDPDGYEFLEILKQVARDNTDNPDLSILWIDPDDFPL
LVAYWEKTFKIDLFRPQIGVVNVTDADSVWMEIPDDDDLPTAEELEDWI
EDVLSGKINTEDDDEDDDDDD

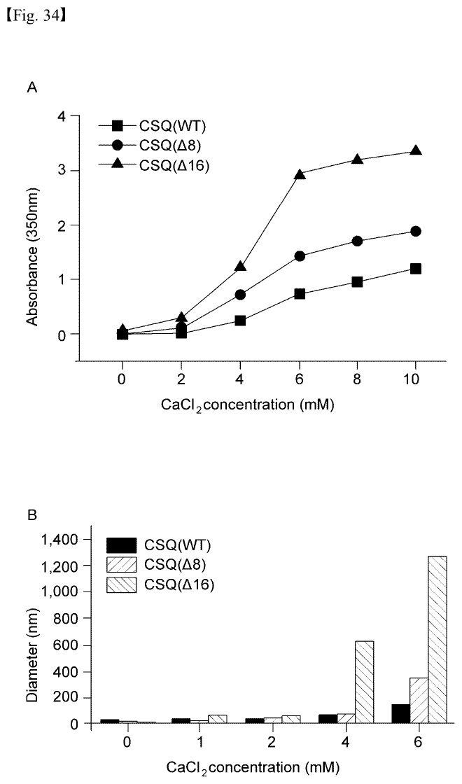

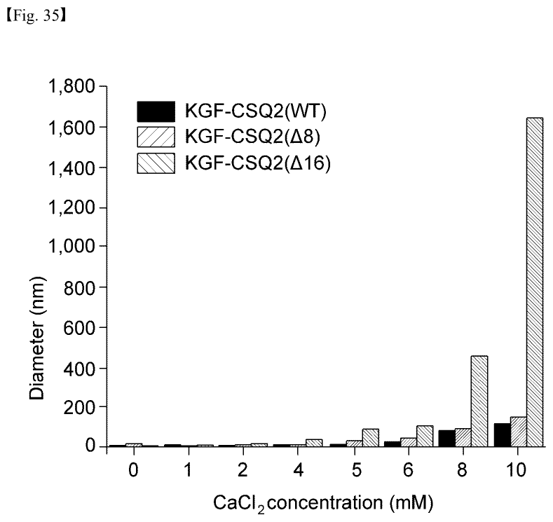

The sizes of the resulting KGF-CSQ2(WT), KGF-CSQ2(Δ8), and KGF-CSQ2 (Δ16) particles were then measured using fluorescence microscopy and dynamic light scattering. The results confirmed that the loss of the C-terminal acidic tail of calsequestrin allows particles to form at lower metal ion concentrations (FIGS. 34-35).

### [17. Preparation and Characterization of Metal Ion-Particulated CSQ-Cleavable Linker-KGF]

The CSQ-cleavable linker-KGF was prepared using the same method as described in Example 1 above, except that the CSQ-MMP2 cleavable vector was used instead of the CSQ-MCS vector, and CaCl2 was added at a concentration of 10 mM. The amino acid sequence information of the cleavable linker is as follows:

### Cleavable Linker (Sequence #26):

### PLGVRG

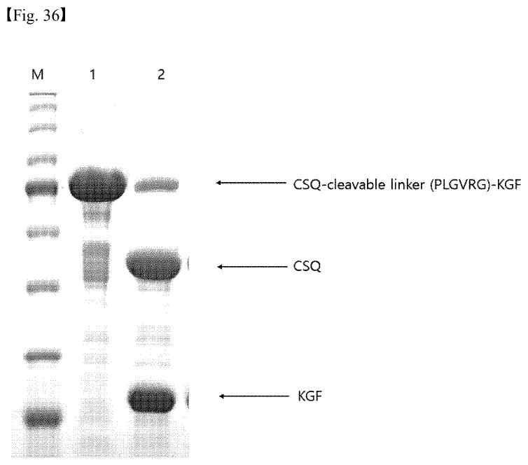

Then, to determine whether the CSQ-PLGVRG-KGF particles generated with 10 mM calcium ions were cleaved by MMP2 to release KGF. 5 μL of MMP2 (Thermo Fisher) was added and reacted at 30° C. for 1 day, followed by analysis using SDS-PAGE. The results confirmed that KGF could be readily cleaved from CSQ-PLGVRG-KGF particles by MMP2 (FIG. 36).

### [18. Preparation and Characterization of Metal Ion-Particulated CSQ-Cleavable Linker-hGH]

CSQ-cleavable linker-hGH was prepared using the same method as described in Example 14, with the exception that the CSQ-MMP2 cleavable vector was used in place of the CSQ-MCS vector, and CaCl2) was added at a concentration of 10 mM. The amino acid sequence information of the cleavable linker is as follows:

### Cleavable Linker (Sequence #26):

### PLGVRG

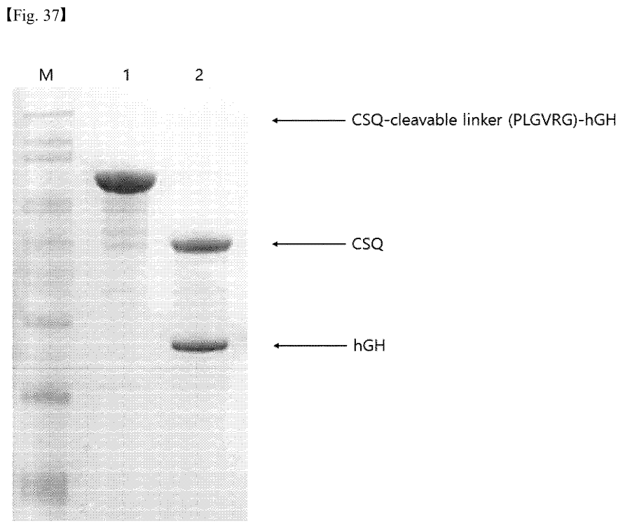

To determine whether the CSQ-PLGVRG-hGH particles generated with 10 mM calcium ions were cleaved by MMP2 to release hGH, 5 μL of MMP2 (Thermo Fisher) was added and incubated at 30° C. for 1 day. The samples were then analyzed by SDS-PAGE. The results confirmed that hGH could be readily cleaved from CSQ-PLGVRG-hGH particles by MMP2 (FIG. 37).

### [19. Preparation and Characterization of Conjugation of Metal Ion-Particulated Exenatide-CSQ]

The peptide with N-hydroxysuccinimide (NHS) attached to the exenatide peptide was synthesized by Anygen. Exenatide-NHS was reacted with CSQ at a 10-fold molar ratio for 6 hours. The exenatide-CSQ conjugate was then purified using a desalting column.

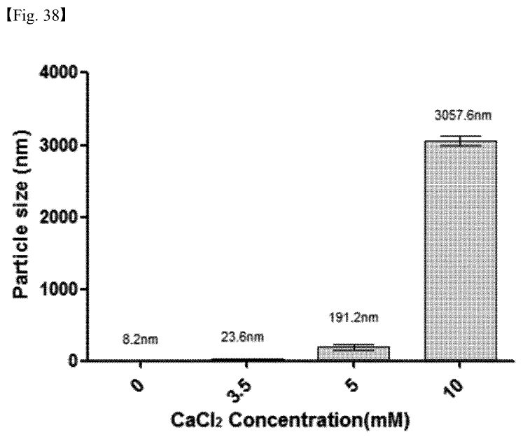

Calcium ions were added in concentrations ranging from 0 to 10 mM to the previously generated exenatide-CSQ conjugate. After a 30 minute reaction at room temperature, the resulting particle size was measured using dynamic light scattering photometry. The results confirmed that the exenatide-CSQ conjugate reacted with the metal ions to form stable particles (FIG. 38).

[20. Conjugation Preparation and Characterization of Metal Ion Particulate hGH-CSQ

CSQ protein was reacted with SMCC (succinimidyl 4-(N-maleimidomethyl)cyclohexane-1-carboxylate) at a 10-fold molar ratio to produce CSQ-maleimide. Subsequently, the cysteine residues of hGH were used to react with CSQ-maleimide at a 1:1 molar ratio for 12 hours. The unreacted CSQ-maleimide and hGH were then separated by size exclusion chromatography (SEC), resulting in the isolation of the CSQ-hGH conjugate.

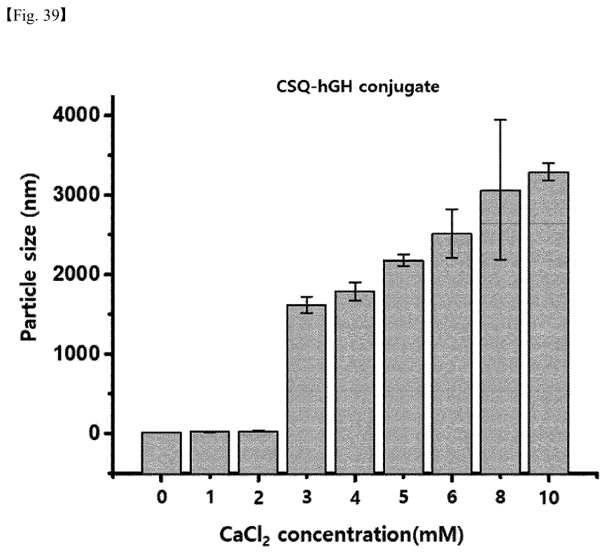

The hGH-CSQ conjugate that was generated previously was then combined with metal ions (calcium) in concentrations ranging from 0 to 10 mM. Following a 30-minute reaction at room temperature, the resulting particle size was measured using dynamic light scattering photometry. The results demonstrated that the hGH-CSQ conjugate reacts with metal ions, forming stable particulates (FIG. 39).

### INDUSTRIAL AVAILABILITY

The calsequestrin based metal ion-reactive particles of the present invention offer enhanced stability due to the inhibition of degradation by hydrolytic enzymes in the body, compared to their pre-particle state. The half-life in the body is extended, which can improve the persistence of the effect of the bioactive substance. Furthermore, the in vitro stability of the bioactive substance is improved, which can maintain its activity and increase the delivery efficiency of the antigen.

1.-69. (canceled)

70. A particle containing:
calsequestrin (CSQ);
a bioactive substance linked to the CSQ; and
a metal ionically linked to CSQ.

71. The particle according to claim 70, wherein the metal comprises at least one selected from the group consisting of zinc, calcium, magnesium, iron, copper, and manganese.

72. The particle according to claim 70, wherein the metal excludes calcium.

73. The particle according to claim 70, wherein the bioactive substance linked to the calsequestrin comprises a cleavage site between the bioactive substance and the calsequestrin.

74. The particle according to claim 70, wherein the bioactive substance linked to the calsequestrin is not cleaved between the bioactive substance and the calsequestrin.

75. The particle according to claim 70, wherein the calsequestrin comprises an amino acid sequence having at least 85% sequence homology to includes one or more amino acid sequences that are selected from the group consisting of SEQ ID NO: 1-11.

76. The particle according to claim 70, wherein the calsequestrin consist of a sequence selected from the group consisting of SEQ ID NO: 1-11.

77. The particle according to claim 70, wherein the bioactive substance comprises at least one selected from the group consisting of polymeric proteins, peptides, glycoproteins, cytokines, growth factors, blood products, vaccines, hormones, enzymes, antibodies, and synthetic agents.

78. The particle according to claim 70, wherein the bioactive substance comprises at least one selected from the group consisting of interleukin-2, blood factor VII, blood factor VIII, blood factor IX, immunoglobulin, horseradish peroxidase (HRP), cytokines, α-interferon, β-interferon, γ-interferon, granulocyte-macrophage colony-stimulating factor (GM-CSF), human fibronectin extra domain B (EBD), fibroblast growth factor (FGF), nerve growth factor (NGF), insulin-like growth factor (IGF), transforming growth factor-α and -β (TGF-α, -β), brain-derived neurotrophic factor (BDNF), platelet-derived growth factor (PDGF), placental growth factor (PlGF), hepatocyte growth factor (HGF), exendin, somatostatin, luteinizing hormone-releasing hormone (LHRH), adrenocorticotropic hormone, growth hormone-releasing hormone, oxytocin, thymosin alpha-1, corticotropin-releasing factor, calcitonin, bivalirudin, vasopressin, phospholipase-activated protein (PLAP), insulin, tumor necrosis factor (TNF), gonadotropin-releasing hormone, thyroid-stimulating hormone, antidiuretic hormone, melanocyte-stimulating hormone, parathyroid hormone, luteinizing hormone, calcitonin gene-related peptide (CGRP), enkephalin, somatomedin, erythropoietin, hypothalamic releasing factors, prolactin, chronic gonadotropin, tissue plasminogen activator, growth hormone-releasing peptide (GHRP), thymic humoral factor (THF), asparaginase, arginase, arginine deaminase, adenosine deaminase, superoxide dismutase, endotoxinase, catalase, chymotrypsin, lipase, uricase, adenosine diphosphatase, tyrosinase, bilirubin oxidase, glucose oxidase, glucosidase, galactosidase, glucocerebrosidase, glucuronidase, keratinocyte growth factor (KGF), exenatide, doxorubicin, anti-vascular endothelial growth factor (Anti-VEGF scFv), SARS-COV-2 receptor binding domain (RBD), epidermal growth factor (EGF), fibroblast growth factor-2 (FGF-2), bone morphogenetic protein (BMP2), transforming growth factor (TGF), vascular endothelial growth factor (VEGF), glucagon-like peptide-1 (GLP-1), TEV protease, HRP enzyme, human growth hormone (hGH), and granulocyte colony-stimulating factor (G-CSF).

79. The particle according to claim 70, wherein the particle has an average diameter from 10 nm to 1 cm.

80. The particle according to claim 70 for use in delivering the bioactive substance to a subject in need thereof.

81. The particle according to claim 70 for use in tissue regeneration.

82. A pharmaceutical composition comprising the particle of claim 70 for preventing or treating at least one disease selected from the group consisting of skin wounds, diabetes, inflammatory diseases, ophthalmic diseases, tissue regeneration, infectious diseases, and cancer.

83. A vaccine comprising the particle of claim 70 for preventing or treating at least one disease selected from the group consisting of inflammatory diseases, infectious diseases, and cancer.

84. The particle according to claim 70, wherein a half-life of the bioactive substance is increased compared to a corresponding bioactive substance not in a particle form.

85. The particle according to claim 70, wherein a stability of the bioactive substance in a body is increased compared to a corresponding bioactive substance not in a particle form.

86. The particle according to claim 70, wherein an activity maintenance is increased compared to a corresponding bioactive substance not in a particle form by increasing a stability of the bioactive substance in a body.

87. A method of delivering a bioactive substance to a subject in need thereof, comprising administering the calsequestrin (CSQ), the bioactive substance and the metal to form the particle of claim 70 in the subject.

88. The particle according to claim 87, further comprising releasing the bioactive substance from the particle as a free form.

89. The particle according to claim 87, further comprising releasing the bioactive substance from the particle as being linked to the CSQ.


SEQUENCE LISTING (26 sequences)

SEQ ID NO: 1 - protein, 362 aa, synthetic construct
QEGLDFPEYDGVDRVINVNAKNYKNVFKKYEVLALLYHEPPEDDKASQRQFEMEELILELAAQVLEDKGVGFGLVDSEKDAAVAKKLGLTEVDSMYVFKGDEVIEYDGEFSADTIVEFLLDVLEDPVELIEGERELQAFENIEDEIKLIGYFKSKDSEHYKAFEDAAEEFHPYIPFFATFDSKVAKKLTLKLNEIDFYEAFMEEPVTIPDKPNSEEEIVNFVEEHRRSTLRKLKPESMYETWEDDMDGIHIVAFAEEADPDGFEFLETLKAVAQDNTENPDLSIIWIDPDDFPLLVPYWEKTFDIDLSAPQIGVVNVTDADSVWMEMDDEEDLPSAEELEDWLEDVLEGEINTEDDDDDDDD

SEQ ID NO: 2 - protein, 380 aa, synthetic construct
EEGLNFPTYDGKDRVVSLSEKNFKQVLKKYDLLCLYYHEPVSSDKVTPKQFQLKEIVLELVAQVLEHKAIGFVMVDAKKEAKLAKKLGFDEEGSLYILKGDRTIEFDGEFAADVLVEFLLDLIEDPVEIISSKLEVQAFERIEDYIKLIGFFKSEDSEYYKAFEEAAEHFQPYIKFFATFDKGVAKKLSLKMNEVDFYEPFMDEPIAIPNKPYTEEELVEFVKEHQRPTLRRLRPEEMFETWEDDLNGIHIVAFAEKSDPDGYEFLEILKQVARDNTDNPDLSILWIDPDDFPLLVAYWEKTFKIDLFRPQIGVVNVTDADSVWMEIPDDDDLPTAEELEDWIEDVLSGKINTEDDDEDDDDDDNSDEEDNDDSDDDDDE

SEQ ID NO: 3 - protein, 372 aa, synthetic construct
EEGLNFPTYDGKDRVVSLSEKNFKQVLKKYDLLCLYYHEPVSSDKVTPKQFQLKEIVLELVAQVLEHKAIGFVMVDAKKEAKLAKKLGFDEEGSLYILKGDRTIEFDGEFAADVLVEFLLDLIEDPVEIISSKLEVQAFERIEDYIKLIGFFKSEDSEYYKAFEEAAEHFQPYIKFFATFDKGVAKKLSLKMNEVDFYEPFMDEPIAIPNKPYTEEELVEFVKEHQRPTLRRLRPEEMFETWEDDLNGIHIVAFAEKSDPDGYEFLEILKQVARDNTDNPDLSILWIDPDDFPLLVAYWEKTFKIDLFRPQIGVVNVTDADSVWMEIPDDDDLPTAEELEDWIEDVLSGKINTEDDDEDDDDDDNSDEEDND

SEQ ID NO: 4 - protein, 364 aa, synthetic construct
EEGLNFPTYDGKDRVVSLSEKNFKQVLKKYDLLCLYYHEPVSSDKVTPKQFQLKEIVLELVAQVLEHKAIGFVMVDAKKEAKLAKKLGFDEEGSLYILKGDRTIEFDGEFAADVLVEFLLDLIEDPVEIISSKLEVQAFERIEDYIKLIGFFKSEDSEYYKAFEEAAEHFQPYIKFFATFDKGVAKKLSLKMNEVDFYEPFMDEPIAIPNKPYTEEELVEFVKEHQRPTLRRLRPEEMFETWEDDLNGIHIVAFAEKSDPDGYEFLEILKQVARDNTDNPDLSILWIDPDDFPLLVAYWEKTFKIDLFRPQIGVVNVTDADSVWMEIPDDDDLPTAEELEDWIEDVLSGKINTEDDDEDDDDDD

SEQ ID NO: 5 - protein, 371 aa, synthetic construct
EDGLDFPEYDGVDRVINVNAKNYKNVFKKYEVLALLYHEPPEDDKASQRQFEMEELILELAAQVLEDKGVGFGLVDSEKDAAVAKKLGLTEEDSVYVFKGDEVIEYDGEFSADTLVEFLLDVLEDPVELIEGERELQAFENIEDEIKLIGYFKSKDSEHYKAYEDAAEEFHPYIPFFATFDSKVAKKLTLKLNEIDFYEAFMEEPMTIPDKPNSEEEIVSFVEEHRRSTLRKLKPESMYETWEDDLDGIHIVAFAEEADPDGYEFLETLKAVAQDNTENPDLSIIWIDPDDFPLLVPYWEKTFDIDLSAPQIGVVNVTDADSIWMEMDNEEDLPSADELEDWLEDVLEGEINTEDDDDDDDDDDDDDDDDD

SEQ ID NO: 6 - protein, 396 aa, synthetic construct
EEGLNFPTYDGKDRVVSLSEKNLKQMLKRYDLLCLYYHEPVSSDKVSQKQFQLKEIVLELVAQVLEHKNIGFVMVDSRKEAKLAKRLGFSEEGSLYVLKGDRTIEFDGEFAADVLVEFLLDLIEDPVEIVNNKLEVQAFERIEDQTKLLGFFKNEDSEYYKAFQEAAEHFQPYIKFFATFDKAVAKKLSLKMNEVGFYEPFMDEPNVIPNKPYTEEELVEFVKEHQRPTLRRLRPEDMFETWEDDLNGIHIVAFAEKSDPDGYEFLEILKQVARDNTDNPDLSILWIDPDDFPLLVAYWEKTFKIDLFKPQIGVVNVTDADSIWMEIPDDDDLPTAEELEDWIEDVLSGKINTEDDDNEDEDDDGDDNDDDDDDDDDNDNSDEDNEDSDDDDDDDE

SEQ ID NO: 7 - protein, 367 aa, synthetic construct
EEGLEFPEYDGVDRVVNVNAKNYKNVFKKYEVLALLYHEPPEDDKASQRQFEMEELILELAAQVLEDKGVGFGLVDSEKDAAVAKKLGLTEEDSIYVFKGDEVIEYDGEFSADTLVEFLLDVLEDPVELIEGERELQAFENIEDEIKLIGYFKNKDSEHYKAFEDAAEEFHPYIPFFATFDSKVAKKLTLKLNEIDFYEAFMEEPVTIPDKPNSEEEIVSFVEEHRRSTLRKLKPESMYETWEDDMDGIHIVAFAEEADPDGFEFLETLKAVAQDNTDNPDLSIIWIDPDDFPLLVPYWEKTFDIDLSAPQIGVVNVTDADSVWMEMDDEEDLPSAEELEDWLEDVLAGEINTEDDDEEEDDDDDDD

SEQ ID NO: 8 - protein, 391 aa, synthetic construct
EEGLNFPTYDGKDRVVSLTEKNFKQVLKKYDVLCLYYHESVSSDKVAQKQFQLKEIVLELVAQVLEHKDIGFVMVDAKKEAKLAKKLGFDEEGSLYVLKGDRTIEFDGEFAADVLVEFLLDLIEDPVEIINSKLEVQAFERIEDQIKLIGFFKSEESEYYKAFEEAAEHFQPYIKFFATFDKGVAKKLSLKMNEVDFYEPFMDEPIAIPDKPYTEEELVEFVKEHQRPTLRRLRPEDMFETWEDDLNGIHIVAFAERSDPDGYEFLEILKQVARDNTDNPDLSIVWIDPDDFPLLVAYWEKTFKIDLFKPQIGVVNVTDADSVWMEIPDDDDLPTAEELEDWIEDVLSGKINTEDDDNEEGDDGDDDEDDDDDDGNNSDEESNDDSDDDDE

SEQ ID NO: 9 - protein, 366 aa, synthetic construct
EDGLDFPEYDGVDRVVNVNAKNYKNIFKKYEVLALLYHEPPEDDKASQRQFEMEELILELAAQVLEDKGVGFGLVDSEKDAAVAKKLGLTEEDSIYVFKGDEVIEYDGEFSADTLVEFLLDVLEDPVELIEGERELQAFENIEDEIKLIGYFKSKDSEHYKAYEDAAEEFHPYIPFFATFDSKVAKKLTLKLNEIDFYEAFMEEPVTIPDKPNSEEEIVSFVEEHRRSTLRKLKPESMYETWEDDMDGIHIVAFAEEADPDGYEFLETLKSVAQDNTDNPDLSIIWIDPDDFPLLVPYWEKTFDIDLSAPQIGVVNVTDADSVWLEMDDEEDLPSAEELEDWLEDVLEGEINTEDDDEEEDDDDDD

SEQ ID NO: 10 - protein, 390 aa, synthetic construct
EEGLNFPTYDGKDRVVSLTEKNFKQVLKKYDLLCLYYHEPVSSDKVAQKQFQLKEIVLELVAQVLEHKDIGFVMVDAKKEAKLAKKLGFDEEGSLYVLKGDRTIEFDGEFAADVLVEFLLDLIEDPVEIINSKLEVQAFERIEDHIKLIGFFKSEDSEYYKAFEEAAEHFQPYIKFFATFDKGVAKKLSLKMNEVDFYEPFMEEPIAIPDKPYTEEELVEFVKEHQRPTLRRLRPEDMFETWEDDLNGIHIVAFAERSDPDGYEFLEILKQVARDNTDNPDLSIVWIDPDDFPLLVAYWEKTFKIDLFKPQIGVVNVTDADSVWMEIPDDDDLPTAEELEDWIEDVLSGKINTEDDDNEDEDDRDDDEDDDDDDDDNSDEDNDDSDDDDE

SEQ ID NO: 11 - protein, 395 aa, synthetic construct
NEDLECMFLGYPDLEYDGFDRTEVLTEKNFNRTVFAEDTKSVVFFNDVEEDDSELDQYECFLQLSAQIMTKRGYNFYTVNTTKEHRLRKQEEVEKGEDTIHVYKDGYKIEYNGVRDPETFVSWLMDIPDDPVTIINDEHDLEEFENMDDECVRIIGYFEPGSVALKEFEEAAEDFMGEIEFFAVVTSKWARKVGLKRVGEVQMRRPFEEDPLFAPTSADTEEEFEDWVEKNKEPVMQKLTLDNYFNLWRDPEEEERMILAFVDEETREGRAMKRLLDKIADENSEHAGTLEIILVDPDEFPLMVDVWEDMFGIDIEEGPQIGLIDISEKEGIWFDMSQVNLDDPKKHSDSNFEALQSWIDQILSGSISLDDDDDDEPEPPAPPPTPKGKKSRKEL

SEQ ID NO: 12 - protein, 164 aa, synthetic construct
MCNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 13 - protein, 41 aa, synthetic construct
AAHGEGTFTSDLSKQMEEEAVRLFIEWLKNGGPSSGAPPPS

SEQ ID NO: 14 - protein, 201 aa, synthetic construct
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 15 - protein, 53 aa, synthetic construct
NSDSECPLSHDGYCLHDGVCMYIEALDKYACNCVVGYIGERCQYRDLKWWELR

SEQ ID NO: 16 - protein, 251 aa, synthetic construct
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 17 - protein, 146 aa, synthetic construct
PALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 18 - protein, 115 aa, synthetic construct
MQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 19 - protein, 113 aa, synthetic construct
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCSR

SEQ ID NO: 20 - protein, 147 aa, synthetic construct
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 21 - protein, 32 aa, synthetic construct
AAHGEGTFTSDVSSYLEEQAAKEFIAWLVKGA

SEQ ID NO: 22 - protein, 236 aa, synthetic construct
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 23 - protein, 323 aa, synthetic construct
QLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSLLHDMVEVVDFVSSM

SEQ ID NO: 24 - protein, 191 aa, synthetic construct [sequence omitted]

SEQ ID NO: 25 - protein, 174 aa, synthetic construct
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 26 - protein, 6 aa, synthetic construct
PLGVRG